(19) United States
(12) Patent Application Publication
(10) Pub. No.: US 2004/0002157 A1
(43) Pub. Date: Jan. 1, 2004

(54) Novel retroviral constructs

(75) Inventors: Johnson Chung-Shun Mak, Victoria (AU); Melissa Kathleen Hill, Victoria (AU); Suzanne Mary Crowe, Victoria (AU)

Correspondence Address: Robert M. O'Keefe, O'KEEFE, EGAN & PETERMAN, Building C, Suite 200, 1101 Capital of Texas Highway South, Austin, TX, 78746, US

(73) Assignee: The MacFarlane Burnet Centre for Medical Research

(21) Appl. No.: 10/255,825

(22) Filed: Sep. 26, 2002

Related U.S. Application Data
(63) Continuation of application No. 09/671,724, filed on Oct. 3, 2002, now abandoned.
Application No. 09/671,724 is a 371 of international application No. PCT/AU99/00219, filed on Mar. 26, 1999.
(60) Provisional application No. 60/079,772, filed on Mar. 27, 1998.

(51) Int. Cl. C12N 15/867; C12N 7/00

(52) U.S. Cl. 435/456; 435/235.1; 435/320.1

(57) ABSTRACT

The present invention relates generally to a method for packaging a gene and genetic constructs useful for same. The method of the present invention represents a new packaging strategy involving vectors comprising retroviral elements and provides for a more efficacious gene therapy and gene modifying protocol. Accordingly, the present invention provides a genetic construct useful as a nucleic acid delivery vector comprising a retroviral packaging signal, a nucleotide sequence derived from viral RNA and which, in RNA form, is translatable to at least one protein and means to facilitate entry of a second nucleotide sequence and wherein said genetic construct, in an RNA form, is capable of being packaged in the presence of retroviral proteins provided in trans with greater efficiency than a genetic construct comprising a packaging signal and said second nucleotide sequence without a nucleotide sequence derived from a retrovirus and which, in RNA form, is capable of translation into a viral protein.

### FIELD OF THE INVENTION

The present invention relates generally to a method for packaging a gene and genetic constructs useful for same. The method of the present invention represents a new packaging strategy involving vectors comprising retroviral elements and provides for a more efficacious gene therapy and gene modifying protocol. Accordingly, the present invention provides a genetic construct useful as a nucleic acid delivery vector comprising a retroviral packaging signal, a nucleotide sequence derived from viral RNA and which in RNA form, is translatable to at least one protein and means to facilitate entry of a second nucleotide sequence and wherein said genetic construct, in an RNA form, is capable of being packaged in the presence of retroviral proteins provided in trans with greater efficiency than a genetic construct comprising a packaging signal and said second nucleotide sequence without a nucleotide sequence derived from a retrovirus and which, in RNA form, is capable of translation into a viral protein.

### BACKGROUND OF THE INVENTION

Bibliographic details of the publications numerically referred to in this specification are collected at the end of the description.

The subject specification contains nucleotide and amino acid sequence information prepared using the programme Patent In Version 2.0, presented herein after the bibliography. Each nucleotide or amino acid sequence is identified in the sequence listing by the numeric indicator <210> followed by the sequence identifier (e.g. <210>1, <210>2, etc). The length, type of sequence (DNA, protein (PRT), etc) and source organism for each nucleotide or amino acid sequence are indicated by information provided in the numeric indicator fields <211>, <212> and <213>, respectively. Nucleotide and amino acid sequences referred to in the specification are defined by the information provided in numeric indicator field <400> followed by the sequence identifier (e.g. <400>1, <400>2, etc).

The designation of nucleotide residues referred to herein are those recommended by the IUPAC-IUB Biochemical Nomenclature Commission, wherein A represents Adenine, C represents Cytosine, G represents Guanine, T represents thymine, Y represents a pyrimidine residue. R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

The rapidly increasing sophistication of recombinant DNA technology is greatly facilitating research and development in the medical and allied health fields. One particularly important area is gene therapy and the ability to introduce genetic material into a target genome.

Considerable interest has been directed to retroviruses in the development of gene transfer vectors for potential use in gene therapy Retroviruses comprise a large and diverse family of enveloped RNA viruses. Retroviruses are generally characterised by their structure, composition and replicative properties (1, 2, 3). A particularly significant feature of retroviruses is their replicative strategy which includes reverse transcription of viral RNA into linear double stranded DNA and subsequent integration of this DNA into the genome of a host cell.

The retroviral genome comprises three major coding domains: gag, which directs synthesis of internal viral proteins; pol, which provides the reverse transcriptase and integration enzymes and in some cases a protease; and env, which directs production of surface and transmembrane components of the viral envelope protein.

The feature of retroviruses to integrate into genomic DNA of a host cell with a high degree of efficiency has been exploited in the development of retroviral gene vectors. Of particular importance has been the discovery that the retroviral genome can accommodate extensive alterations including, deletions, substitutions and/or additions of nucleotides. Where such alterations result in an inability to replicate, a replication competent “helper” virus has nevertheless been used to propagate the altered virus. For the purposes of gene therapy, however, the use of helper viruses is not acceptable especially due to potential safety concerns.

Retroviral packaging systems have also been developed in which cells are generated expressing in trans the proteins required for retroviral packaging (4, 5) These cells are referred to as “packaging cells”. One disadvantage, however, of early packaging cells has been the production of helper viruses following recombinational events. Further genetic engineering has assisted in creating vectors where the viral coding regions are so deleted that the possibility of recombination resulting in production of live viruses is reduced (6).

A range of retroviral vectors and packaging cell lines are now available in the scientific community. However, the vectors and packaging cell lines have been developed based on a hypothesis of how packaging occurs in a cell. In essence, it has traditionally been considered that any type of RNA could be packaged by providing a packaging (ψ) signal. This has led to a range of vectors being produced which result in low efficiency packaging.

In work leading up to the present invention, the inventors further investigated viral RNA packaging. They have now surprisingly determined that efficient packaging of RNA requires more than the provision of a ψ signal. In particular, in accordance with the present invention, efficient packaging of RNA requires a ψ signal in combination with viral proteins including specific viral proteins and their precursor proteins. The RNA-protein “complex” is then preferentially packaged within the virion. The elucidation of this packaging mechanism now enables the design of a new range of nucleic acid delivery vectors comprising retroviral elements which permit the efficient packaging of RNA.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

One aspect of the present invention provides a genetic construct useful as a nucleic acid delivery vector comprising a retroviral packaging signal, a nucleotide sequence derived from viral RNA and which, in RNA form, is translatable to at least one protein and means to facilitate entry of a second nucleotide sequence and wherein said genetic construct, in an RNA form, is capable of being packaged in the presence of retroviral proteins provided in trans with greater efficiency than a genetic construct comprising a packaging signal and said second nucleotide sequence without a nucleotide sequence derived from a retrovirus and which, in RNA form, is capable of translation into a viral protein.

Another aspect of the present invention is directed to a genetic construct useful as a nucleic acid delivery vector comprising a nucleotide sequence derived from viral RNA and which, in RNA form, is translatable to at least one protein or derivative thereof encoded by all or part of the gag region of a retrovirus and means to facilitate entry of a second nucleotide sequence.

More particularly, the present invention provides a genetic construct useful as a nucleic acid delivery vector comprising a nucleotide sequence derived from viral RNA and which, in RNA form, is translatable to at least one protein or derivative thereof encoded by all or part of the gag region of a retrovirus and means to facilitate entry of a second nucleotide sequence and wherein said genetic construct, in an RNA form, is capable of being packaged in the presence of retroviral proteins provided in trans with greater efficiency than a genetic construct comprising a packaging signal and said second nucleotide sequence without a nucleotide sequence derived from a retrovirus and which, in RNA form, is capable of translation into at least one protein or derivative thereof encoded by the gag region.

Still another aspect of the present invention relates to a genetic construct useful as a nucleic acid delivery vector comprising a retroviral packaging signal, a nucleotide sequence derived from viral RNA and which, in RNA form, is translatable to at least one protein encoded by the gag region of a retrovirus and at least one protein encoded by the pol region of a retrovirus and a means to facilitate entry of a second nucleotide sequence.

More particularly, the present invention contemplates a genetic construct useful as a nucleic acid delivery vector comprising a retroviral packaging signal, a nucleotide sequence derived from viral RNA and which, in RNA form, is translatable to at least one protein encoded by the gag region of a retrovirus and at least one protein encoded by the pol region of a retrovirus and means to facilitate entry of a second nucleotide sequence and wherein said genetic construct, in an RNA form, is capable of being packaged in the presence of retroviral proteins provided in trans with greater efficiency than a genetic construct comprising a packaging signal and said second nucleotide sequence without a nucleotide sequence derived from a retrovirus and which, in RNA form, is incapable of translation into at least one protein from each of the gag and pol regions.

Yet still another aspect of the present invention provides a genetic construct comprising a retroviral packaging signal, a nucleotide sequence derived from all or a functional part of the gag and pol regions of a retrovirus and means to facilitate entry of a second nucleotide sequence, i.e. a gene of interest to be packaged.

In yet another aspect of the present invention, there is provided a nucleic acid packaging system comprising at least one vector wherein a first vector comprises a retroviral packaging signal, a nucleotide sequence derived from viral RNA and which, in RNA form, is translatable to at least one protein and means to facilitate entry of a second nucleotide sequence and wherein said genetic construct, in an RNA form, is capable of being packaged in the presence of retroviral proteins provided in trans with greater efficiency than a genetic construct comprising a packaging signal and said second nucleotide sequence without a nucleotide sequence derived from a retrovirus and which, in RNA form, is capable of translation into a viral protein: said retroviral packaging system further comprising at least one other vector or packaging cell wherein said vector or cell's genome comprises at least one other retroviral coding region or a functional derivative thereof.

Another aspect of the present invention contemplates a nucleic acid packaging system comprising at least one vector wherein a first vector comprises a retroviral packaging signal, a nucleotide sequence derived from viral RNA and which, in RNA form, is translatable to at least one protein and means to facilitate entry of a second nucleotide sequence and wherein said genetic construct, in an RNA form, is capable of being packaged in the presence of retroviral proteins provided in Trans with greater efficiency than a genetic construct comprising a packaging signal and said second nucleotide sequence without a nucleotide sequence derived from a retrovirus and which, in RNA form, is capable of translation into a viral protein; said retroviral packaging system further comprising at least one other vector or packaging cell wherein said vector of cell's genome comprises the env region of a virus or a functional equivalent or derivative thereof.

Still another aspect of the present invention is directed to a nucleic acid packaging system comprising a first and second vector wherein the first vector is a nucleic acid delivery vector and comprises a retroviral packaging signal, a nucleotide sequence derived from viral RNA and which, in RNA form, is translatable to at least one protein encoded by the gag region of a retrovirus and means to facilitate entry of a second nucleotide sequence; wherein said second vector comprises the env region of a virus or a functional equivalent or derivative thereof.

More particularly, the present invention provides a nucleic acid packaging system comprising a nucleic acid delivery vector having a retroviral packaging signal, a nucleotide sequence derived from viral RNA and which, in RNA form, is translatable to at least one protein encoded by the gag region of a retrovirus and means to facilitate entry of a second nucleotide sequence and wherein said genetic construct, in an RNA form, is capable of being packaged in the presence of retroviral proteins provided in trans with greater efficiency than a genetic construct comprising a packaging signal and said second nucleotide sequence without a nucleotide sequence derived from a retrovirus and which, in RNA form, is capable of translation into a protein encoded by the gag binding region of a retrovirus gag protein; and at least one other vector comprising the env coding region of a virus or a functional equivalent or derivative thereof.

Another aspect of the present invention contemplates a method for packaging a nucleic acid, said method comprising introducing said nucleic acid into a nucleic acid delivery vector comprising a retroviral packaging signal and a nucleotide sequence derived from viral RNA and which, in RNA form, is translatable to at least one protein and wherein said genetic construct, in an RNA form, is capable of being packaged in the presence of retroviral proteins provided In trans with greater efficiency than a genetic construct comprising a packaging signal and said nucleic acid without a nucleotide sequence derived from a retrovirus and which, in RNA form, is capable of translation into a viral protein, introducing said nucleic acid delivery vector into a cell wherein said cell is capable of providing the genetic material or a protein encoded thereby in trans to permit packaging of said nucleic acid into a virion.

Yet another aspect of the present invention relates to a method for packaging a nucleic acid of interest, said method comprising introducing said nucleic acid into a nucleic acid delivery vector comprising a retroviral packaging signal, gag or a functionally equivalent region thereof and optionally pol or a functionally equivalent region thereof corresponding to pol and introducing said nucleic acid delivery vector into a cell which contains or is capable of containing genetic material or a protein encoded thereby to permit packaging of said nucleic acid of interest into a virion.

Preferably, the proteins provided in trans to permit packaging are encoded by nucleotide sequences contained in a second vector.

Still a further aspect of the present invention is directed to a gene delivery vector in DNA form comprising all or a functional part of the following:

(i) two retroviral long terminal repeat sequences (LTR) at the two ends of the retroviral DNA genome,

(ii) a primer binding site sequence (PBS);

(iii) a retroviral genomic RNA packaging sequence (ψ) or equivalent;

(iv) a nucleotide sequence derived from RNA and which, in an RNA form, is translatable to at least one protein;

(v) optionally, a DNA sequence encoding a transcriptional enhancer element;

(vi) a retroviral Rev responsive element or its equivalent;

(vii) a second nucleotide sequence which consists of a genetic construct that is used for gene delivery into the target cells (e.g. see FIG. 15); and

(viii) optionally, a gene of interest expression control element.

Yet still another aspect of the present invention provides a tropism determining vector comprising all or a functional part of the following:

(i) a nucleotide sequence, in an RNA form, is translatable to viral envelope proteins as the envelope of a gene delivery vector;

(ii) One or more nucleotide sequences, in RNA form, translatable to lentiviral auxiliary proteins Vif, Vpr and/or Nef or their equivalents; and/or

(iii) a nucleotide sequence, in an RNA form, translatable to HIV-1 Rev protein or its equivalent.

A further aspect of the present invention provides for the use of gag or a functionally equivalent region thereof in the manufacture of a nucleic acid delivery vector capable of carrying a nucleic acid of interest to be packaged into a virion.

### BRIEF DESCRIPTION OF THE FIGURES

FIG. 1 is a diagrammatic representation of retroviral genomic RNA.

FIG. 2 is a diagrammatic representation showing co-transfection system using a gag+rev− (Rev−) vector and a gag−rev+ (Env or MA_{UAA}).

FIG. 3 is a diagrammatic representation of possible outcomes following packaging of gag+rev− (Rev−) and gag−rev+ (MA_{UAA}) mutants.

FIG. 4 is a photographic representation showing analysis of cellular expression of viral protein from gag+rev− (Rev−) and gag−rev+ (Env or MA_{UAA}) mutants.

FIG. 5 contains two parts, which are the diagrammatic and photographic representations showing northern analysis of virion genomic RNA.

FIG. 6 is a diagrammatic representation of the HIV-1 proviral DNA genome.

FIG. 7 is a diagrammatic representation of the M5 gene delivery vector.

FIG. 8 is a diagrammatic representation of the tropism determining vector.

FIG. 9 is a diagrammatic representation of the M5 gene delivery vector with highlight of the LTR region.

FIG. 10 is a diagrammatic representation of the M5 gene delivery vector with highlight of the PBS sequences.

FIG. 11 is a diagrammatic representation of the M5 gene delivery vector with highlight of the ψ sequences.

FIG. 12 is a diagrammatic representation of the M5 gene delivery vector with highlight of the Gag and Gag-Pol sequences.

FIG. 13 is a diagrammatic representation of the M5 gene delivery vector with highlight of the tat sequences.

FIG. 14 is a diagrammatic representation of the M5 gene delivery vector with highlight of the RRE sequences.

FIG. 15 is a diagrammatic representation of the M5 gene delivery vector with highlight of the gene of interest.

FIG. 16 is a diagrammatic representation of the M5 gene delivery vector with highlight of the expression control elements.

FIG. 17 is a diagrammatic representation of the tropism determining vector with highlight of the env sequences.

FIG. 18 is a diagrammatic representation of the tropism determining vector with highlight of the lentiviral auxiliary protein coding sequences.

FIG. 19 is a diagrammatic representation of the tropism determining vector with highlight of the rev sequences.

FIG. 20 is a diagrammatic representation of the prototypes of M5 gene delivery vector (A) and tropism determining vector (B) used in the present study. The prototype M5 gene delivery vector contains Gag, Gag-pol coding sequence, RRE coding sequence and green floresence protein (EGFP) as the gene of interest. The prototype tropism determining vector is essentially the second half of the HIV-1 genome that encodes for Env, Rev, Tat and HIV-1 auxiliary proteins (i.e. Vif, Vpr, Vpu and Nef).

FIG. 21 is a diagrammatic representation of conventional retroviral gene delivery vector (A) and a plasmid DNA that provides viral proteins (B), for retroviral gene delivery vector packaging cell line viral protein expression plasmid.

FIG. 22 is a photographic representation of the quantitative polymerase chain reaction (PCR) for cellular HLA-DQ a sequence-normalization of cellular DNA input for the comparison of gene delivery efficiency between conventional gene delivery vector and prototype of M5 gene delivery vector.

FIG. 23 is a photographic representation of the quantitative polymerase chain reaction (PCR) for EGFP cDNA synthesis using three different populations of donor PBMC as the target cells for gene delivery.

FIG. 24 is a diagrammatic representation of an HIV-1 or HIV-2 based M5 gene delivery vector (A) and tropism determining vector (B) containing RRE element and transcription enhancer element.

FIG. 25 is a diagrammatic representations showing a MoMuLV based M5 gene delivery vector (A) and a VSV-G envelope based tropism determining vector (B).

FIG. 26 is a diagrammatic representation showing an HIV-1 based M5 gene delivery vector (A) with internal expression control element and tropism determining vector (B) containing Rev.

FIG. 27 is a diagrammatic representation showing an HIV-1 based M5 gene delivery vector (A) with internal conditional expression control element and tropism determining vector (B) containing Rev.

FIG. 28 is a diagrammatic representation of a MoMuLV based M5 gene delivery vector (A) with internal expression control element and self inactivated LTR and a VSV-G envelope based tropism determining vector (B).

FIG. 29 is a diagrammatic representation showing a MoMuLV based M5 gene delivery vector (A) with internal conditional expression control element and self inactivated LTR and a VSV-G envelope based tropism determining vector (B).

FIG. 30 is a diagrammatic representation showing a MoMuLV based M5 gene delivery vector with internal conditional expression control element and self inactivated LTR and a hepatitis virus envelope based tropism determining vector (B).

FIG. 31 is a diagrammatic representation showing a MoMuLV based M5 gene delivery vector (A) with internal conditional expression control element and self inactivated LTR and a hepatitis virus envelope based tropism determining vector (B).

FIG. 32 is a diagrammatic representation showing a MoMuLV based M5 gene delivery vector (A) with internal conditional expression control element and self inactivated LTR and a hepatitis virus envelope based tropism determining vector (B).

### DETAILED DESCRIPTION OF THE PREFERRED EMBODIMENTS

The present invention is predicated in part on the determination that retroviral genomic RNA used for protein synthesis is packaged preferentially into the virion during viral assembly. This is counter to the previously held belief, and the basis of previously designed retroviral delivery vectors, that any RNA would be packaged in the presence of a packaging signal.

Accordingly, one aspect of the present invention provides a genetic construct useful as a nucleic acid delivery vector comprising a retroviral packaging signal, a nucleotide sequence derived from viral RNA and which, in RNA form, is translatable to at least one protein and means to facilitate entry of a second nucleotide sequence and wherein said genetic construct, in an RNA form, is capable of being packaged in the presence of retroviral proteins provided in trans at a higher frequency than a genetic construct comprising a packaging signal and said second nucleotide sequence without a nucleotide sequence derived from a retrovirus and which, in RNA form, is capable of translation into a viral protein.

The first nucleotide sequence derived from a viral genome is one which encodes or is translatable to at least one viral protein. Generally, although not intending to limit the present invention to any one theory or mode of action, the viral protein is capable of “chaperoning” the second nucleotide sequence (referred to herein as a “heterologous sequence”, “gene of interest” or “nucleic acid of interest”) thereby forming a “complex” which is then packaged. Preferably, the first nucleotide sequence corresponds to all or part of the gag coding region or a derivative or homologue thereof. A “derivative” of gag includes single or multiple nucleotide substitutions, deletions and/or additions to the gag sequence. Generally, a derivative gag sequence still encodes sufficient Gag proteins to induce packaging of the second nucleotide sequence.

Reference herein to “gag” includes reference to the gag coding region which encodes all or some of the Gag proteins such as matrix protein (MA), capsid protein (CA) and nucleocapsid protein (NC). A “Gag” protein may be an individual protein or it may refer to two or more Gag proteins such as MA, CA and/or NC. Reference to “Gag” also includes fusion molecules comprising Gag such as Gag-Pro, Gag-Pol or Gag-Pro-Pol or derivatives or homologues thereof. Corresponding genetic fusions are also contemplated by the present invention, i.e. gag-pro, gag-pol and gag-pro-pol or derivatives or homologues thereof.

Reference herein to “pol” means a coding region for viral reverse transcriptase. Generally, although not universally, the coding region encodes a Gag-Pro-Pol precursor polyprotein. Reference herein to “pol” includes any homologues and derivatives thereof including genetic fusions such as gag-pro, gag-pol and gag-pro-pol.

The pro coding region encodes a viral protease. In many cases, the pro open reading frame (ORF) is −1 with respect to the gag ORF and a Gag-Pro polypeptide is expressed by a −1 frameshift during translation. Reference to “pro” or Pro includes homologues, derivatives and fusions thereof.

The expression “means to facilitate entry” of a second nucleotide sequence is used in its broadest context and includes such means as homologous recombination, site-directed mutagenesis, site-directed transposition, blunt-end ligation, restriction endonuclease digestion followed by ligation, random transposon-like insertion amongst other means. The most convenient means, and preferred in accordance with the present invention, is the presence of at least one restriction endonuclease site which is first cleaved. The second nucleotide sequence is then ligated into the cleaved site.

According to this preferred embodiment, there is provided a genetic construct useful as a nucleic acid delivery vector comprising a retroviral packaging signal, a nucleotide sequence derived from viral RNA and which, in RNA form, is translatable to at least one protein and a restriction endonuclease site to facilitate entry of a second nucleotide sequence and wherein said genetic construct, in an RNA form, is capable of being packaged in the presence of retroviral proteins provided in trans with greater efficiency than a genetic construct comprising a packaging signal and said second nucleotide sequence without a nucleotide sequence derived from a retrovirus and which, in RNA form, is capable of translation into a viral protein.

The term “greater efficiency” is also used in its broadest context and may be determined qualitatively or quantitatively. In essence, in accordance with the present invention, more viral particles will be produced which will contain packaged nucleic acid and which will be infective. Consequently, the efficiency of packaging is conveniently but not exclusively determined with reference to a particle infectivity ratio. The ratio will, in accordance with the present invention, be lower compared to a packaging system based on a retroviral packaging signal and nucleic acid of interest without retroviral derived nucleic acid translatable into at least one retroviral protein. This is due to an increase in the number of infective viral particles.

Accordingly, it is proposed, in accordance with the present invention that following packaging, P_{N} viral particles will be produced of which P_{I} will be infective. Generally, P_{N}>P_{I} and P_{N}/P_{I} is the particle:infectivity ratio, R_{I}.

It is proposed that P_{I}, following the method of the present invention (P_{I(inv)}) will be higher than P_{I} using the more classical method (P_{I(inv)})

Accordingly, P_{N}/P_{I(inv)}<P_{N}/P_{I(cla)} occurs when there is greater efficiency as proposed in accordance with the present invention.

Accordingly, another embodiment of the present invention contemplates a genetic construct useful as nucleic acid delivery vector comprising a retroviral packaging signal, a nucleotide sequence derived from viral RNA and which, in RNA form, is translatable to at least one protein and means to facilitate entry of a second nucleotide sequence and wherein said genetic construct, in RNA form, is capable of being packaged in the presence of retroviral proteins provided in trans such that the ratio P_{N}/P_{I(inv)} wherein P_{N} is the number of viral particles produced of which P_{I(inv)} are infective, is lower then the ratio P_{N}/P_{I(cla)}, wherein P_{N} is as defined above and P_{I(cla)} is the number of infective viral particles produced by a genetic construct comprising a packaging signal and said second nucleotide sequence without a nucleotide sequence derived from a retrovirus and which, in RNA form, is capable of translation into a viral protein.

In an alternative, efficiency of packaging may also be determined by the number of viral particles produced which are infective and are capable of introducing a nucleic acid molecule of interest into the Genome of a host cell. Efficiency can then be measured by the expression of the nucleic acid molecule of interest.

Reference herein to the “second nucleotide sequence” means, as stated above, a nucleotide sequence not naturally associated, that is, not normally contiguous with a viral derived nucleotide sequence encoding at least one viral protein. In this sense, the second nucleotide sequence may be considered to be “heterologous.” although this is not to imply that the second nucleotide sequence cannot be another retroviral derived nucleic acid molecule.

Another aspect of the present invention is directed to a genetic construct useful as a nucleic acid delivery vector comprising a nucleotide sequence derived from viral RNA and which, in RNA form, is translatable to at least one protein or derivative thereof encoded by all or part of the gag region of a retrovirus and means to facilitate entry of a second nucleotide sequence.

More particularly, the present invention provides a genetic construct useful as a nucleic acid delivery vector comprising a nucleotide sequence derived from viral RNA and which, in RNA form, is translatable to at least one protein or derivative thereof encoded by all or part of the gag region of a retrovirus and means to facilitate entry of a second nucleotide sequence and wherein said genetic construct, in an RNA form, is capable of being packaged in the presence of retroviral proteins provided in trans with greater efficiency than a genetic construct comprising a packaging signal and said second nucleotide sequence without a nucleotide sequence derived from a retrovirus and which, in RNA form, is capable of translation into at least one protein or derivative thereof encoded by the gag region.

Generally, the retroviral packaging signal is present on the genetic construct. The packaging signal and the nucleotide sequence derived from RNA and which, in RNA form, is translatable to at least one protein such as a Gag protein may be contiguous with each other in the sense that both regions are derived from a single piece of viral RNA or may be derived from separate regions of viral RNA, from the same or different viruses, but ligated or fused together. A retroviral packaging signal is referred to as “ψ” and may be the entire region from a retrovirus or a functional derivative thereof.

The gag region and packaging signal may be from the same virus or from different strains or species of retrovirus.

Preferably, the present invention provides a nucleic acid delivery vector comprising elements from any retrovirus. Examples of retroviruses from which vector elements may be derived include but are not limited to viruses from the A vain sarcoma and leukosis viral group (e.g. Rouse Sarcoma Virus), mammalian B-type viral group (e.g. Mouse Mammary Tumor Virus), murine leukemia related viral croup (e.g. Moloney Murine Leukemia Virus), human T-cell leukemia/bovine leukemia viral group (e.g. Human T-Cell Leukemia Virus), D-type viral group (e.g. Mason-Pfizer Monkey Virus), Lentiviruses (e.g. Human Immuno-Deficiency Virus) and Spumaviruses (e.g. Human Foamy Virus). The nucleic acid delivery vector of the present invention may comprise genetic elements from a single retrovirus or may contain genetic elements for two or more different retroviruses. In a particularly preferred embodiment, the nucleic acid delivery vector is regarded as a retroviral delivery vector.

In a particularly preferred embodiment of the present invention, the portion of the genetic construct which is derived from a viral RNA genome capable of translation into a protein, is capable of translation into one or more Gag proteins and one or more Pol proteins.

Even more preferably, the entire gag, pol and/or pro coding regions are represented in the genetic construct or at least functional parts or derivatives or homologues thereof.

Still another aspect of the present invention relates to a genetic construct useful as a nucleic acid delivery vector comprising a retroviral packaging signal, a nucleotide sequence derived from viral RNA and which, in RNA form, is translatable to at least one protein encoded by the gag region of a retrovirus and at least one protein encoded by the pol region of a retrovirus and means to facilitate entry of a second nucleotide sequence.

More particularly, the present invention contemplates a genetic construct useful as a nucleic acid delivery vector comprising a retroviral packaging signal, a nucleotide sequence derived from viral RNA and which, in RNA form, is translatable to at least one protein encoded by the gag region of a retrovirus and at least one protein encoded by the pol region of retrovirus and means to facilitate entry of a second nucleotide sequence and wherein said genetic construct, in an RNA form, is capable of being packaged in the presence of retroviral proteins provided in trans with greater efficiency than a genetic construct comprising a packaging signal and said second nucleotide sequence without a nucleotide sequence derived from a retrovirus and which, in RNA form, is capable of translation into at least one protein for each of the gag and pol regions.

In still a further preferred aspect of the present invention, there is provided a genetic construct comprising a retroviral packaging signal, nucleotide sequences derived from all or a functional part of the gag and pol regions of a retrovirus and means to facilitate entry of a nucleic acid of interest, to be packaged.

Reference to a “functional part” in accordance with this aspect of the present invention means that the retroviral-derived nucleotide sequences encode or are translatable into a sufficient amount of active Gag proteins and Pol proteins to effect packaging in the presence of other retroviral genes provided in trans with greater efficiency than if the nucleic acid of interest is provided with a retroviral packaging signal but without the gag and/or pol encoding regions. A “nucleic acid of interest” and a “gene of interest” may be used interchangedly throughout this specification although the term “gene” should be considered in its broadest context to include any nucleotide sequence which, in RNA form, is translatable to any amino acid sequence or which acts as an anti-sense molecule or ribozyme.

The preferred construct of the present invention is prepared by deleting the env and aux genes from the retrovirus genome and the nucleic acid of interest is generally located 3′ of the pol gene. The advantage of removing the env and aux coding regions from the retroviral delivery vector includes a reduced chance of immune response to these molecules if such a construct is used in gene therapy. Furthermore, it is preferable to remove all or part of rev from the retroviral delivery vector while retaining a functionally active part of RRE. This has the effect of trapping Gag and/or Gag-Pol mRNAs in the nucleus post delivery of the nucleic acid of interest. This also has the effect of minimising an immune response to these molecules. It is preferable, in accordance with the present invention, that the host cells be immunologically as “silent” as possible to prevent an immune response being activated against such cells or proteins produced by such cells. In this regard, where a nucleic acid delivery molecule does not include env or aux genes, there is a reduced risk that host cells will become an immunological target. Similar considerations apply to using rev nucleic acid delivery vectors since Gag and Gal-Pol will be trapped within the nucleus.

The genetic construct of the present invention may be in single or double stranded RNA (e.g. mRNA) or DNA (e.g. cDNA) form in linear or covalently closed circular form. Diploid RNA and RNA/DNA hybrids are also contemplated by the present invention. Providing the genetic construct in DNA form is preferred due to stability, ease of handling and ease of genetic manipulation. However, single stranded or double stranded RNA molecules may also be used. Such molecules generally require conversion to double stranded DNA by reverse transcription prior to use. Such conversion may occur in vitro or in vivo.

The genetic constructs of the present invention are particularly useful as part of a retroviral packaging system. In one retroviral packaging system contemplated by the present invention, two or more vectors are generally provided wherein at least one vector is the nucleic acid delivery vector as described herein and at least one other of the vectors contains the env gene or a functional equivalent or derivative thereof and/or other genes encoding viral proteins required from packaging. Alternatively, the retroviral packaging system comprises a nucleic acid delivery vector and a packaging cell line comprising viral genes expressed from its genome. The env gene is conveniently from a retrovirus although the present invention contemplates the use of env nucleotide sequence from non-retroviral sources to facilitate targeting of a nucleic acid delivery vector to particular cell types. For example, an env nucleotide sequence from Hep B virus will target a hybrid viral particle to hepatic cells. Where components are derived from non-retroviral sources, the vector system is still referred to as a retroviral packaging system or a retroviral delivery vector. The env gene sequence may also come from non-viral sources such as cellular sources and this is encompassed by a “functional equivalent”.

In yet another aspect of the present invention, there is provided a retroviral packaging system comprising at least one vector wherein a first vector comprises a retroviral packaging signal, a nucleotide sequence derived from viral RNA and which, in RNA form, is translatable to at least one protein and a means to facilitate entry of a second nucleotide sequence and wherein said genetic construct, in an RNA form, is capable of being packaged in the presence of retroviral proteins provided in trans with greater efficiency than a genetic construct comprising a packaging signal and said second nucleotide sequence without a nucleotide sequence derived from a retrovirus and which, in RNA form, is capable of translation into a viral protein; said retrovirus packaging system further comprising at least one other vector or packaging cell wherein said vector or cell's genome comprises at least one other retroviral coding region or a functional derivative thereof.

More particularly, the present invention contemplates a retroviral packaging system comprising at least one vector wherein a first vector comprises a retroviral packaging signal, a nucleotide sequence derived from viral RNA and which, in RNA form, is translatable to at least one protein and means to facilitate entry of a second nucleotide sequence and wherein said genetic construct, in an RNA form, is capable of being packaged in the presence of retroviral proteins provided in trans with greater efficiency than a genetic construct comprising a packaging signal and said second nucleotide sequence without a nucleotide sequence derived from a retrovirus and which, in RNA form, is capable of translation into a viral protein: said retrovirus packaging system further comprising at least one other vector or packaging cell wherein said vector or cell's genome comprises the env region of a virus or a functional equivalent or derivative thereof.

One skilled in the art will immediately recognise that the retroviral packaging systems of the present invention may be modified. For example, the system may contain a single vector comprising a nucleic acid delivery vector and the other viral genes required for packaging provided in trans in the genome of a packaging cell. Alternatively, two or more vectors may be employed. All such variations are encompassed by the present invention and do not fall outside the scope of a nucleic acid delivery vector as herein contemplated.

In a particularly preferred embodiment, the present invention provides a retroviral packaging system comprising a first and second vector wherein the first vector is a nucleic acid delivery vector and comprises a retroviral packaging signal, a nucleotide sequence derived from viral RNA and which, in RNA form, is translatable to at least one protein encoded by the gag region of a retrovirus and a restriction endonuclease site to facilitate entry of a second nucleotide sequence; wherein said second vector comprises the env region of a virus or a functional equivalent or derivative thereof.

Even more particularly, the present invention is directed to a retroviral packaging system comprising a nucleic acid delivery vector having a retroviral packaging signal, a nucleotide sequence derived from viral RNA and which, in RNA form, is translatable to at least one protein encoded by the gag region of a retrovirus and to facilitate entry of a second nucleotide sequence and wherein said genetic construct, in an RNA form, is capable of being packaged in the presence of retroviral proteins provided in trans with greater efficiency than a genetic construct comprising a packaging signal and said second nucleotide sequence without a nucleotide sequence derived from a retrovirus and which, in RNA form, is capable of translation into a protein encoded by the gag coding region of a retrovirus; and at least one other vector comprising the env coding region of a virus or a functional equivalent or derivative thereof.

Preferably, the nucleic acid delivery vector encodes at least one Gag protein and at least one Pol protein or functional derivatives thereof.

Even more preferably, the nucleic acid delivery vector comprises the full retroviral wild-type gag and pol regions of one or more retroviruses.

Still more preferably, the nucleic acid delivery vector encodes a Rev responsive element (RRE) but does not contain a functional rev.

One particularly preferred embodiment of the present invention is described below.

The genetic construct useful as a nucleic acid delivery vector comprising a retroviral packaging signal, a nucleotide sequence derived from viral RNA and which, in RNA form, is translatable to at least one protein and means to facilitate entry of a second nucleotide sequence and wherein said genetic construct, in an RNA form, is capable of being packaged in the presence of retroviral proteins provided in trans, is referred to as the “M5 gene delivery vector”. The vector which provides retroviral protein in trans for the production of gene delivery retroviral particles via the M5 gene delivery vector is referred to as the “tropism determinating vector”.

The gene delivery retroviral particles produced by the expression of both the M5 gene delivery vector and the tropism determining vector are more efficient in the packaging of genomic RNA and more efficient in gene delivery.

In a particularly preferred embodiment, the gene delivery vector in DNA form comprises all or a functional part of the following for efficient gene delivery:

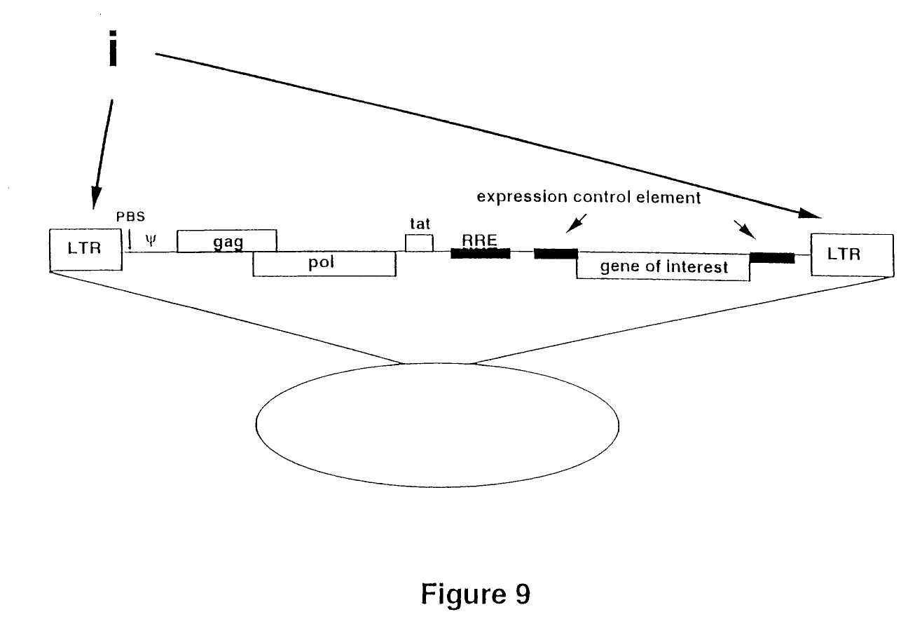

(i) two retroviral long terminal repeat sequences (LTR) at the two ends of the retroviral DNA genome which are required for retroviral RNA expression. In an optional embodiment, a small deletion in the 3′LTR U3 region is also included to generate a self inactive lentivirus vector, post gene delivery (e.g. see FIGS. 9 and 28-32);

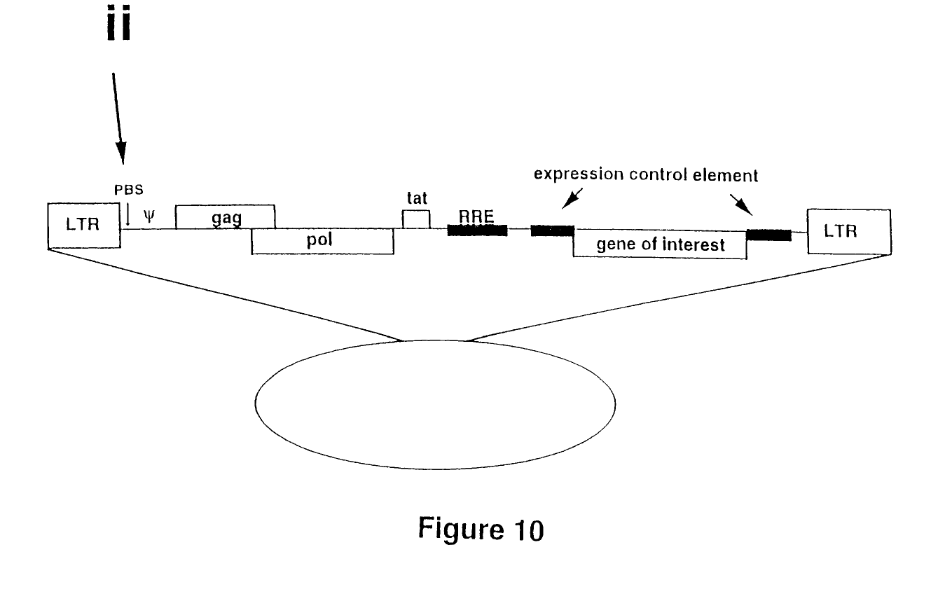

(ii) a primer binding site sequence (PBS) or equivalent required for the binding of retroviral primer tRNA for the synthesis of retroviral cDNA post viral entry into the target cells (e.g. see FIG. 10);

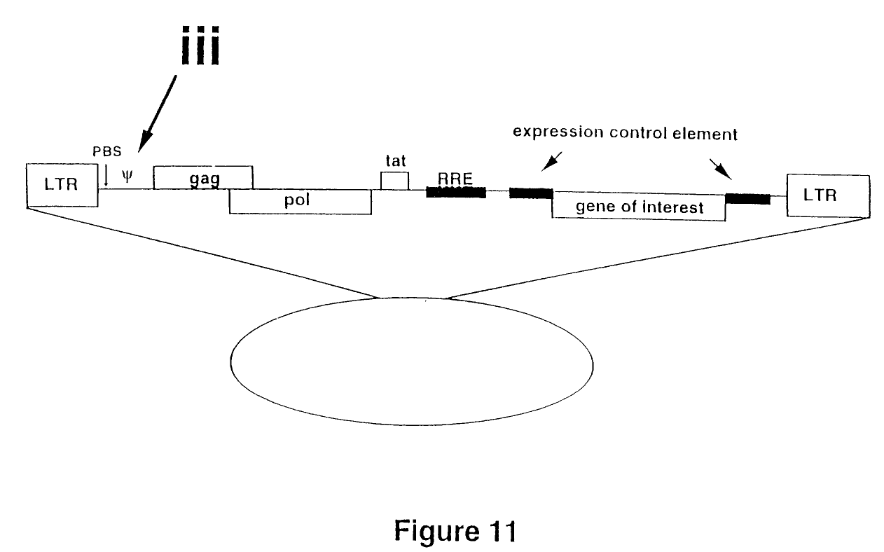

(iii) a retroviral genomic RNA packaging sequence (ψ) or equivalent which is required for the packaging of genomic RNA during viral assembly (e.g. see FIG. 11);

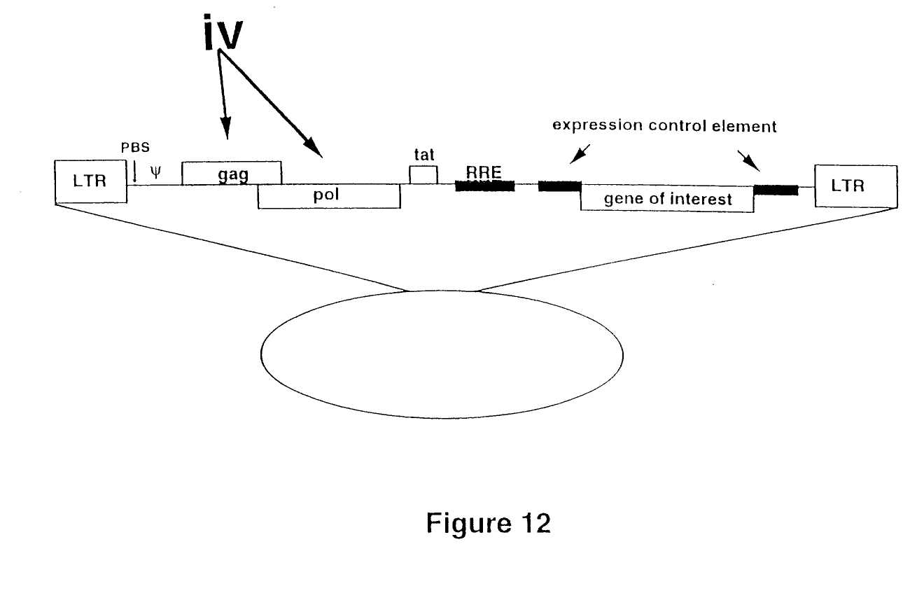

(iv) a nucleotide sequence derived from RNA and which, in an RNA form, is translatable to at least one protein (such as Gag, Gag-Pol and/or part of the retroviral Gag protein) [e.g. see FIG. 12];

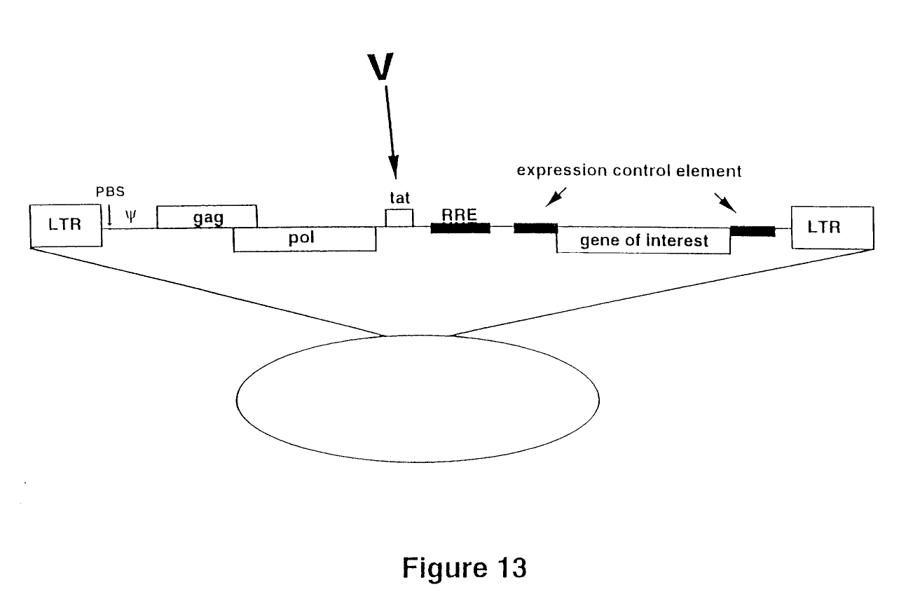

(v) optionally, a DNA sequence encoding a transcriptional enhancer element which is required to enhance the retroviral LTR driven transcription (e.g. see FIG. 13);

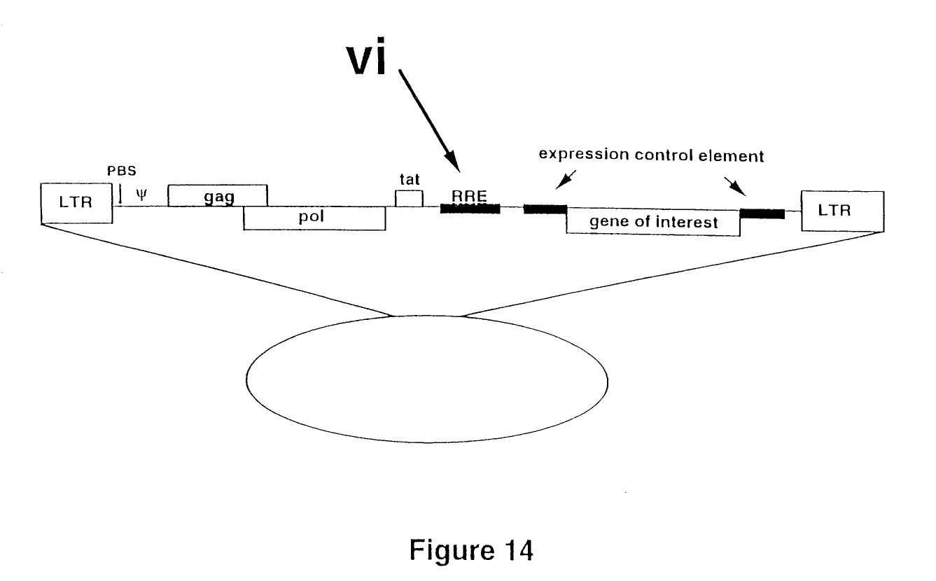

(vi) a retroviral Rev responsive element (or equivalent) which is required for the nuclear retention of unspliced and singly spliced RNA in the absence of Rev protein (or equivalent) [e.g. see FIG. 14];

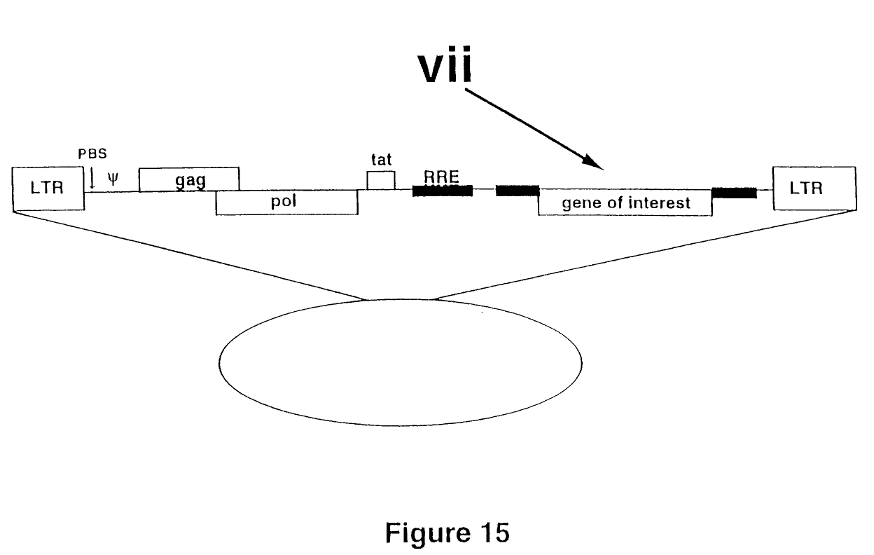

(vii) a second nucleotide sequence (i.e. gene of interest) which consists of a genetic construct that is used for gene delivery into the target cells (e.g. see FIG. 15); and/or

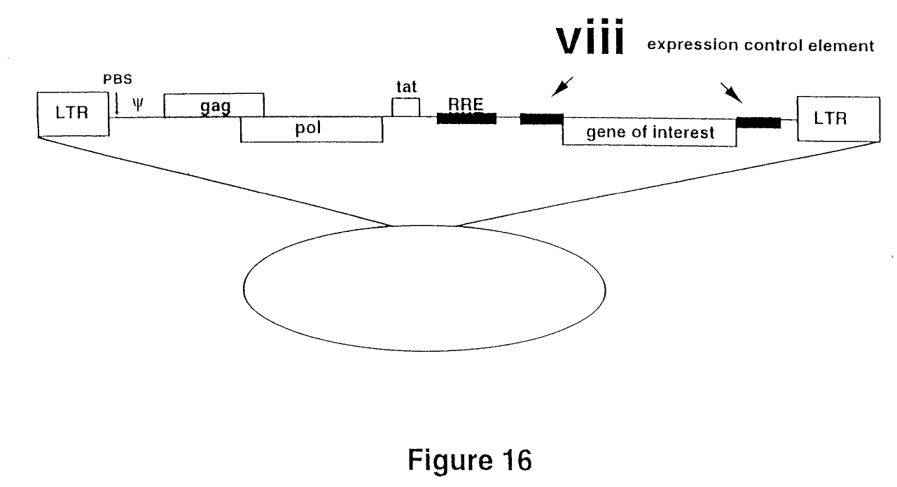

(viii) optionally, a gene of interest expression control element, such as a promoter sequence (which can be placed 5′ of the gene of interest) and/or a translational control sequence (which can be placed 3′ of the gene of interest) in order to enhance and/or for the conditional expression of gene of interest post gene delivery (e.g. see FIG. 16).

In its most preferred embodiment, the gene delivery vector is the M5 gene delivery vector.

The preferred tropism determining vector consists of all or a functional part of the following for the production of retroviral particles and efficient gene delivery:

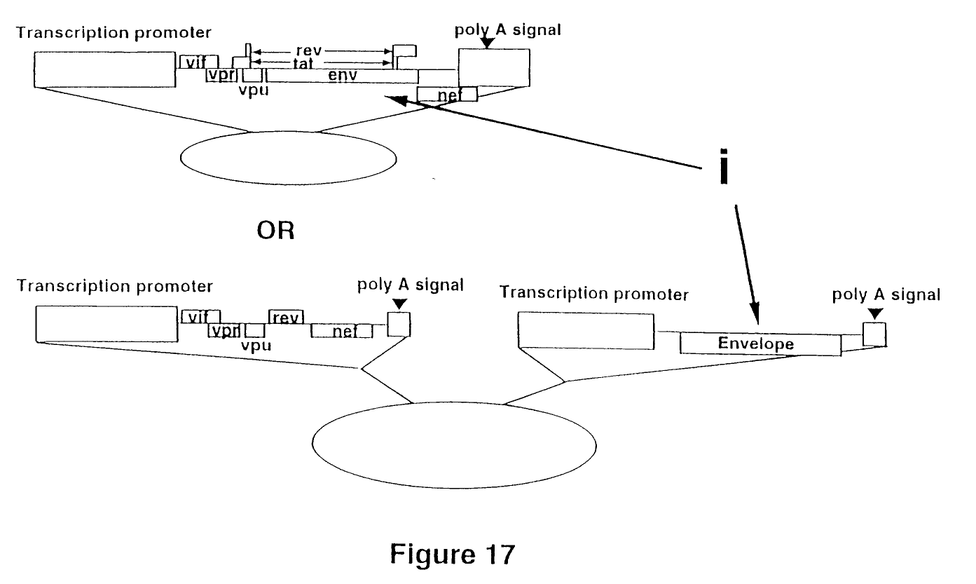

(i) a nucleotide sequence, in an RNA form, translatable to a viral envelope protein as the envelope of the gene delivery vector (e.g. the M5 gene delivery vector) [see FIG. 17];

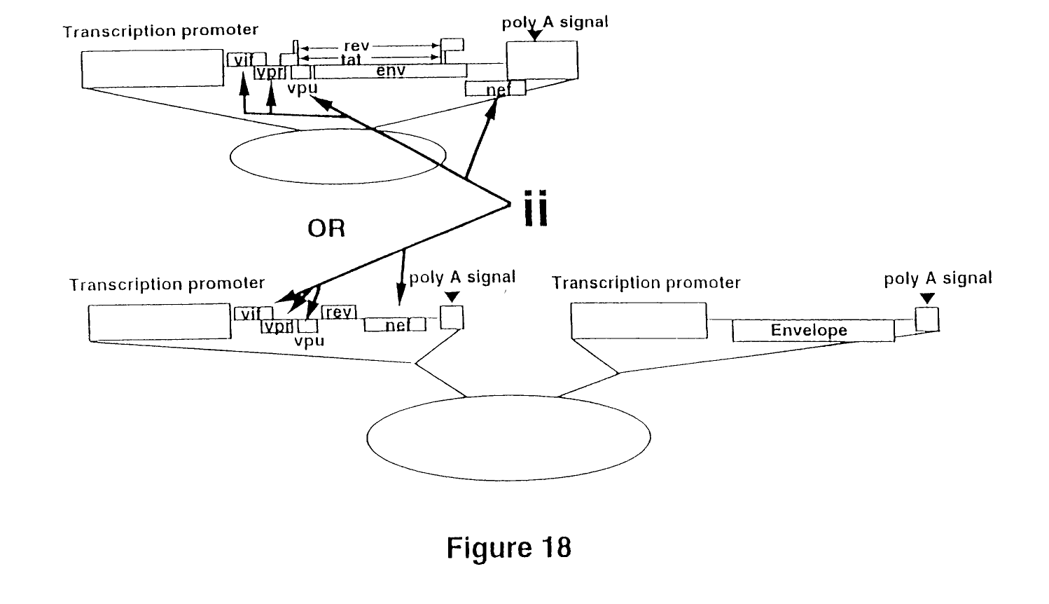

(ii) one or more nucleotide sequences, in RNA form, translatable to lentiviral auxiliary proteins Vif, Vpr and/or Nef and/or their equivalents or functional derivatives to enhance the gene delivery efficiency of the gene delivery vector (e.g. the M5 gene delivery vector) [see FIG. 18]; and/or

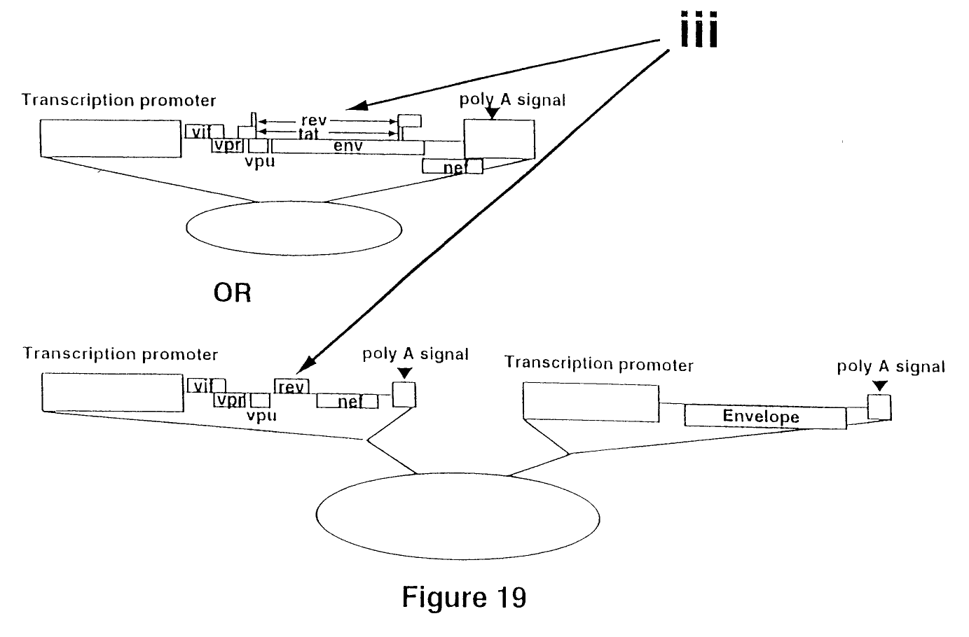

(iii) a nucleotide sequence, in an RNA form, translatable to lentiviral Rev protein (or equivalent) to facilitate the nucleus to cytoplasmic transport of the gene delivery vector (e.g. M5) RNA for protein translation and genomic RNA packaging (see FIG. 19).

The LTR sequences may be derived from any one or more of the following retroviruses, i.e. Rous sarcoma virus (RSV), avian myeloblastosis virus (AMV), avian erythroblastosis virus (AEV), Moloney murine leukemia virus (Mo-MuLV), Harvey murine sarcoma virus (Ha-MSV), Abelson murine leukemia virus (A-MuLV), AKR-MuLV. Feline leukemia virus (FeLV), simian sarcoma virus (SSV), reticuloendotheliosis virus (REV), spleen necrosis virus (SNV), mouse mammary tumor virus (MMTV), Mason-Pfizer monkey virus (MPMV), human T-cell leukemia (or lymphotropic) virus (HTLV)-1 and -2, bovine leukemia virus (BLV), human immunodeficiency virus (HIV)-1 and -2, simian immunodeficiency virus (SIV), feline immunodeficiency virus (FIV), bovine immunodeficiency virus (BIV), visna/maedi virus, caprine arthritis-encephalitis virus (CAEV), equine infectious aneima virus (EIAV), simian foamy virus (SFV), human foamy virus (HFV), human spumareetrovirus (HSRV), and feline syncytium-forming virus (FeSV).

The primer binding site sequence (PBS), the retroviral genomic RNA packaging sequence (ψ), the nucleotide sequence, in RNA form, translatable to at least one protein such as Gag, Gag-Pol or part thereof, the transcriptional enhancer element (e.g. HIV-1 Tat, HIV-9 Tat, SIV Tat, HTLV-1 Tat and HTLV-2 Tat) and the retroviral Rev responsive element (RRE) [e.g. HIV-1RRE, HIV-2 RRE, SIV RRE, HTLV-1 rex responsive element, HTLV-L rex responsive cloned at MPMV CTE element] are preferably but not exclusively, derived from the same retroviral genome as the LTR sequences.

The gene of interest consists of a genetic construct which is used for gene delivery into the target cells can be derived from any nucleotide sequence.

The gene of interest expression control element, such as a promoter sequence is placed 5′ of the gene of interest and/or a translational control sequence is placed either 5′ or 3′ of the gene of interest depending on the nature of the control element in order to enhance and/or to facilitate conditional expression of a gene of interest post gene delivery. Examples of promoter sequences include tetracycline responsive promoter, glucocorticoid steroid responsive promoter, ecdysone insect hormone responsive promoter, copper inducible responsive promoter, zinc inducible responsive promoter, cytomegalovirus CMV promoter (e.g. CMV immediate-early gene promoter), SV40 large T antigen or small T antigen responsive promoter, internal ribosome entry element, lac switch expression system. Examples of the translational control sequence as the gene of interest expression control element are iron responsive element/ferretin receptor translation control element, and estrogen receptor responsive element.

Nucleotide sequences encoding viral proteins of the tropism determining vector may be supplied by plasmid DNA via co-transfection with the M5 gene delivery vector plasmid DNA. The tropism determining vector viral proteins may also be provided via a packaging cell line, where gene delivery viral particles can be generated by introducing M5 gene delivery vector into the appropriate tropism determining vector cell line.

The nucleotide sequence, in an RNA form, translatable to viral envelope proteins as the envelope of M5 gene delivery vector may be derived from but not limited to the following virus envelope proteins, such as members of retroviridae (i.e. Rous sarcoma virus (RSV), avian myeloblastosis virus (AMV), avian erythroblastosis virus (AEV), Moloney murine leukemia virus (Mo-MuLV), Harvey murine sarcoma virus (Ha-MSV), Abelson murine leukemia virus (A-MuLV), AKR-MuLV, Feline leukemia virus (FeLV), simian sarcoma virus (SSV), reticuloendotheliosis virus (REV), spleen necrosis virus (SNV), mouse mammary tumor virus (MMTV), Mason-Pfizer monkey virus (MPMV), human T-cell leukemia (or lymphotropic) virus (HTLV)-1 and -2, bovine leukemia virus (BLV), human immunodeficiency virus (HIV)-1 and -2, simian immunodeficiency virus (SIV), feline immunodeficiency virus (FIV), bovine immunodeficiency virus (BIV), visna/maedi virus, caprine arthritis-encephalitis virus (CAEV), equine infectious aneima virus (EIAV), simian foamy virus (SFV), human foamy virus (HFV), human spumareetrovirus (HSRV), and feline syncytium-forming virus (FeSV)), vesicular stomatitis virus (VSV), members of heparnavindae, members of herpesviridae, members of poxviridae, members of orthomyxoviridae, members of togaviridae, members of flaviviridae, members of coronvinrdae, members of paramyxoviridae, members of rhabdoviridae, and members of filoviridae.

The nucleotide sequences, in RNA form, are translatable to lentiviral auxiliary proteins Vif, Vpr, and/or Nef or their equivalents or functional derivatives to enhance the gene delivery efficiency of the M5 gene delivery vector. The origins of viral auxiliary proteins can be from, but not limited to HIV-1, HIV-2, SIV, HTLV-1 and HTLV-2.

The nucleotide sequence, in an RNA form, translatable to HIV-1 Rev protein (or equivalent) facilitates the nucleus to cytoplasmic transport of the M5 gene delivery vector RNA for protein translation and genomic RNA packaging. Examples of HIV-1 equivalent Rev proteins are, but not limited to, HIV-2 Rev, SIV Rev, HTLV-1 Rex, and HTLV-2 Rex.

The nucleic acid delivery vector is contemplated herein with or without a gene of interest therein inserted. The nucleic acid of interest may be a “gene” in the classical sense or may be any nucleotide sequence of interest including a genomic DNA or cDNA or fragments, parts, derivatives thereof or corresponding mRNA such as encoding a peptide, polypeptide or protein. The nucleic acid of interest may also be an anti-sense molecule or encode a ribozyme or deoxyribozyme. The nucleic acid of interest may include its own promoter or it may be inserted downstream of and operably linked to a promoter resident in the nucleic acid delivery vector. In a particularly preferred embodiment, the nucleic acid of interest is a therapeutic molecule such as but not limited to a cytokine, antibody, cancer gene, genetic marker, interleukin molecule, haemopoietic gene, an immunomodulatory gene or other gene of interest. Generally, the gene is useful in gene therapy.

Another aspect of the present invention contemplates a method for packaging a nucleic acid, said method comprising introducing said nucleic acid into a nucleic acid delivery vector comprising a retroviral packaging signal and a nucleotide sequence derived from viral RNA and which, in RNA form, is translatable to at least one protein and wherein said genetic construct, in an RNA form, is capable of being packaged in the presence of retroviral proteins provided in trans with greater efficiency than a genetic construct comprising a packaging signal and said gene without a nucleotide sequence derived from a retrovirus and which, in RNA form, is capable of translation into a viral protein; introducing said nucleic acid delivery vector into a cell wherein said cell is capable of providing the genetic material or a protein encoded thereby in trans to permit packaging of said gene into a virion.

Generally, at least two vectors are used wherein one vector is a nucleic acid delivery vector and at least one other vector carries genetic material, e.g. env, required for packaging. In a particularly preferred embodiment, the retroviral delivery vector comprises gag or a region substantially corresponding to gag and also optionally pol or a region substantially corresponding to pol and the gene of interest.

Yet another aspect of the present invention relates to a method for packaging a nucleic acid of interest, said method comprising introducing said nucleic acid into a nucleic acid delivery vector comprising a retroviral packaging signal, gag or a functionally equivalent region thereof and optionally pol or a functionally equivalent region thereof corresponding to pot and introducing said nucleic acid delivery vector into a cell which contains or is capable of containing genetic material or a protein encoded thereby to permit packaging of said gene of interest into a virion.

Preferably, genetic information required for packaging is provided on at least one plasmid wherein at least one plasmid carries an env region from a retrovirus or a functional equivalent thereof from another virus.

Still a further aspect of the present invention provides for the use of the gag region of a retrovirus or a functionally equivalent region thereof in the manufacture of a nucleic acid delivery vector capable of carrying a nucleic acid of interest to be packaged.

The nucleic acid delivery vector and packaging system of the present invention is contemplated to be useful in gene therapy. For example, the nucleic acid delivery vector may be used to package genetic material designed to replace an endogenous defective gene or to augment an endogenous gene or to introduce material for co-suppression, antisense activity or ribozyme activity. The virion particle carrying the packaged genetic material are then used as a composition such as a pharmaceutical composition to introduce the genetic material to the required host. The nucleic acid delivery vector and packaging system of the present invention is also contemplated to be useful in production of transgenic animals, transgenic plants, genetically modified cells and in inducing genetically modified animals and plants.

The present invention further extends to cell lines expressing a nucleic acid of interest and introduced into said cell line by a nucleic acid delivery vector herein described.

The present invention is further described by the following non-limiting Examples.

### EXAMPLE 1

### Viral Genomic RNA

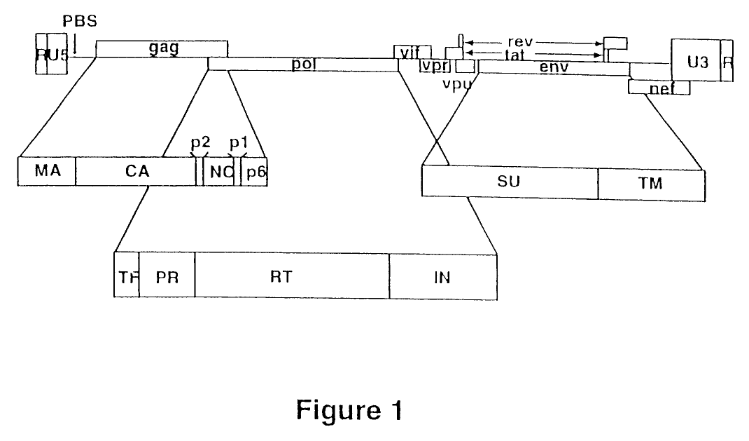

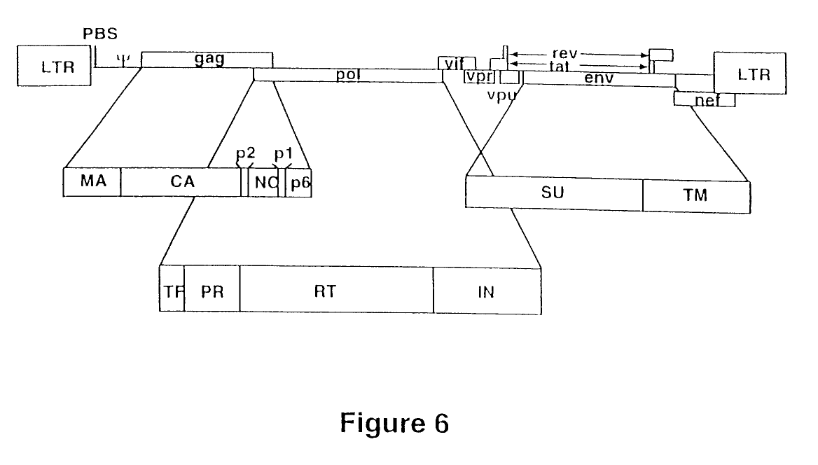

A diagrammatic representation of retroviral genomic RNA is shown in FIG. 1. A DNA form is shown in FIG. 6.

### EXAMPLE 2

### Analysis of Viral Components

Viral components were analysed by introducing proviral DNA with mammalian cells, isolating viral particles and then analysing viral protein and RNA.

### EXAMPLE 3

### Mutant Co-Transfection System

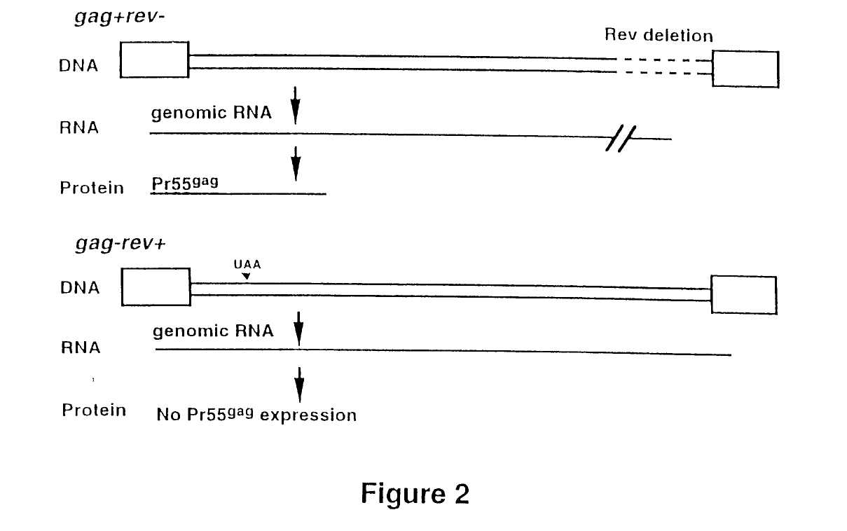

A co-transfection system was developed using two mutant retroviruses. The first was gag⁺rev⁻, carrying a deletion in the rev gene. The second carried an early termination codon immediately after the matrix coding sequence in gag and was gag⁻rev⁺. The mutants are summarized in FIG. 2. Neither retroviral proviral DNAs is able to make viral particles. Both require components developed by the other in order to produce viral particles. In the system shown in FIG. 2, the gag+rev− (Rev−) vector consists of a deletion of the Rev coding sequence at the 3′ of the genomic DNA. In the absence of functional Rev expression, unspliced genomic RNA is trapped in the nucleus and the protein expression of Gag is restricted. Rev is provided by the second vector gag−rev+ (Env or MAUAA). The Env expression vector encodes for the 3′ half of the HIV-1 sequence (such as Tat, Rev. Nef and Env). MAUAA has been engineered with a termination codon immediately 3′ of the matrix (MA) coding sequence which does not allow for the synthesis of full length Gag precursor protein, but MAUAA maintains the ability to synthesize functional Rev protein. Both Gag synthesis and Rev expression are required for the formation of viral particles (HIV-1), hence, viral particles will not be produced when only one of these two plasmid DNA vectors is introduced into the cell, since only one of these two vectors can synthesize Gag protein.

There are, however, two populations of unspliced genomic RNA found in the virion producing cell. Therefore, one can assess which population of unspliced genomic RNA can be packaged into virion (i.e. the population of genomic RNA that synthesize Gag or the population of genomic RNA that does not synthesize Gag).

### EXAMPLE 4

### Reverse Transcriptase (RT) Activity of Co-Transfection System

293T cells and COS cells were transfected with the gag⁺rev⁻ vector or gag⁻rev⁺ vector and assayed for reverse transcriptase (RT) activity. The results are shown in Table 1 and clearly show high RT activity by expressing both proviral DNAs.

### EXAMPLE 5

Analysis of Viral Genomic RNA

Retroviral RNA was detected in cells transfected with the gag⁺rev mutant retrovirus indicating cellular expression of viral RNA.

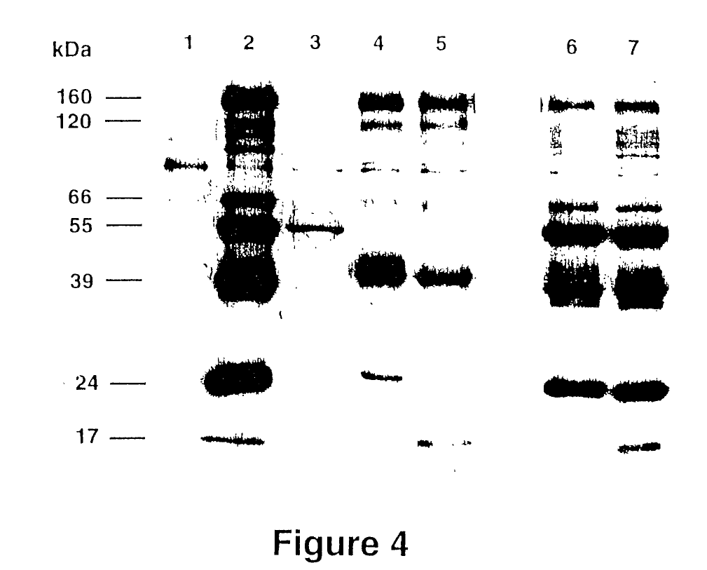

Lane 1 of FIG. 4 is the mock transfection cellular lysate control, and demonstrate the specificity of our viral protein detection system. Lane 2 of FIG. 4 is cellular lysate from cells that are transfected with wild type full length HIV-1 proviral DNA, and it helps to demonstrate all the viral protein that our detection system can detect (i.e. 160 kDa protein—Gag-Pol Pr160 and Env gp160; 120 kDa protein—Env gp120; 66 kDa protein—RT p66; 55 kDa protein—Gag Pr55; 39 to 41 kDa protein—Gag p39 and Env gp41; 24 to 27 kDa protein—CA p24 and Nef p27; and 17 kDa protein—MA p17). FIG. 1 provides the coding regions of various viral proteins. Lane 3 is a cellular lysate from cells that are transfected with Rev− plasmid alone, and only a low level of Gag Pr55 is detected in the subject assay, and RT activity show background level of activity. Lane 4 is cellular lysate from cells that are transfected with Env expression plasmid, and only viral Env and Nef can be detected. Lane 5 is cellular lysate from cells that are transfected with MA_{UAA} plasmid alone, and only viral Env and MA can be detected. Rev− and MA_{UAA} are Nef defective based plasmid, hence, Nef cannot be detected in lanes 3 and 5. Lane 6 is cellular lysate from cells that are transfected with Rev− and Env expression plasmid and lane 7 is cellular lysate from cells that are transfected with Rev− and MA_{UAA} expression plasmid. When both gag+rev− (Rev−) and gag−rev+(Env or MA_{UAA}) plasmid are introduced to the same virus producing cells, the wild type viral protein expression pattern is restored. Lanes 3, 4, 5, 6 and 7 together confirm the design of our co-transfection system, where viral particles can only be produced when both plasmid are Introduced into the same cell. Western analysis is performed using HIV-1 infected patient serum as primary antibody, and horse radish peroxidase conjugated goat anti-human antibody as secondary antibody. Protein bands are visualized by enhanced chemiluminescence assay.

### EXAMPLE 6

### Interaction Between Gag and Genomic RNA During Viral Assembly

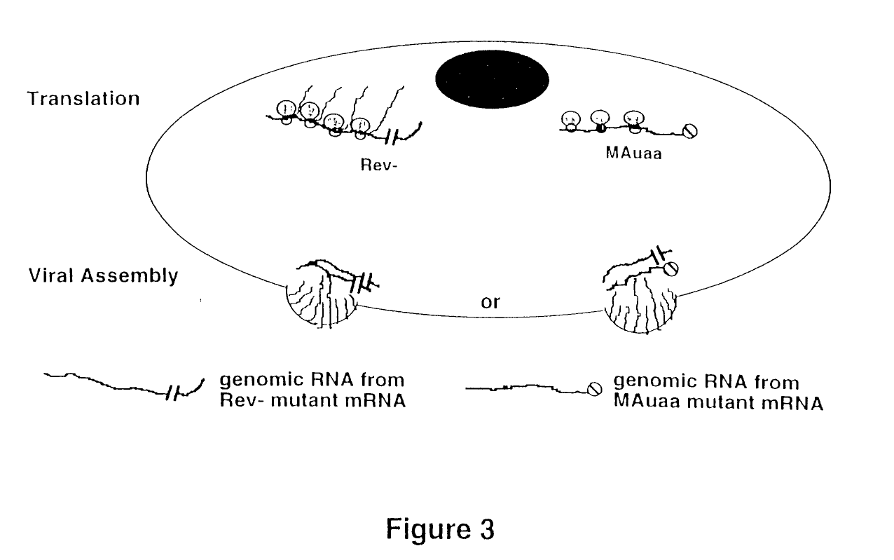

FIG. 3 provides a diagram of the possible genomic composition of viruses packaged following interaction between the gag⁺rev⁻ mutant and the gag rev⁺ mutant retroviruses. The left hand side of FIG. 3 represents that the Rev− mRNA will be preferably packaged into virion (i.e. genomic RNA that is used for Gag protein synthesis is preferably packaged into virion). The right hand side of FIG. 3 represents the more conventional held belief that any genomic RNA contains packaging signal (regardless its ability to synthesize Gag) will have equivalent opportunity to be packaged into virions. In essence, assembled viruses may comprises genomic RNA from both mutant retroviruses if assembly is random (right hand side). Alternatively, if preferential packaging occurs in the presence of Gag, then it would be expected that only gag⁺rev (Rev−) genomic RNA would be represented in the viral particles (left).

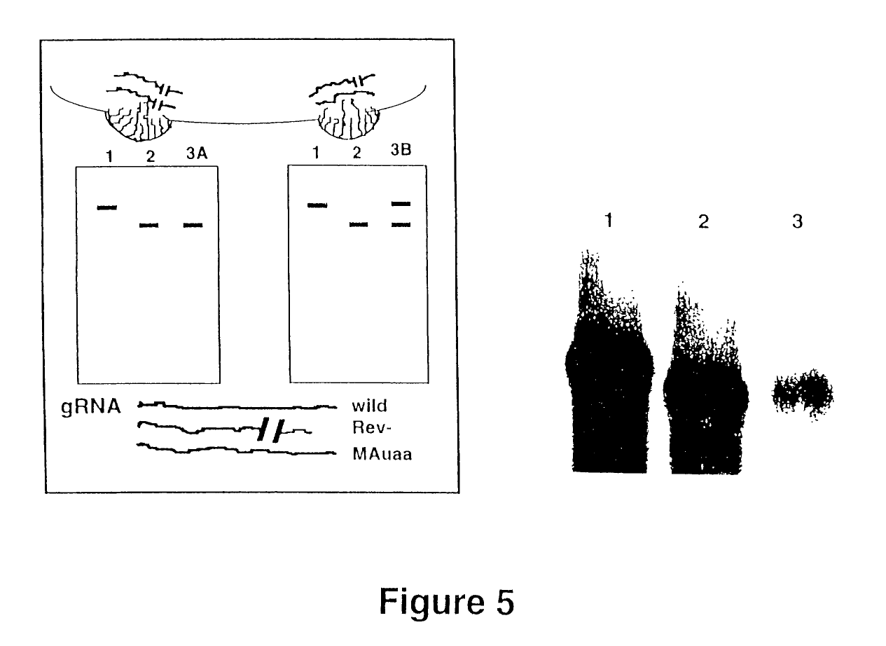

FIG. 5 contains two parts, which are the diagrammatic and photographic representations showing northern analysis of virion genomic RNA. The left hand side is a diagrammatic (hypothetical) representation of possible outcome following packaging of gag⁺rev⁻ (Rev−) and gag rev⁺ (MA_{UAA}) mutants which serves as a reminder for the potential outcome (see FIG. 3 legend for details). The right hand side represents the actual northern analysis of the virion packaged genomic RNA. Lane 1 of the hypothetical northern analysis and the actual northern analysis are full length genomic RNA that are isolated from wild type HIV-1 virions. This provide a size marker control for gag rev⁺ genomic RNA (see lane 2 of FIG. 4 for the western analysis of this transfection). Lane 2 of the hypothetical northern analysis and the actual northern analysis are virion genomic RNA that are isolated from virions that contains only Rev deleted viral genomic RNA. Rev deleted virion genomic RNA provides a size marker control for gag⁺rev⁻ genomic RNA (see lane 6 of FIG. 4 for the western analysis of this transfection). Lane 3A of the hypothetical northern analysis represents the possible outcome if preferential genomic RNA packaging occurs in the presence of Gag during protein translation. Lane 3B of the hypothetical northern analysis represents the possible outcome if virion genomic RNA packaging is independent of Gag synthesis. Lane 3 of the actual northern analysis are virion genomic RNA that are isolated from supernatant collected from 293T cells that have been transfected with our gag⁺rev⁻ (Rev−) and gag⁻rev⁺ (MA_{UAA}) plasmid DNA. The genomic RNA that are found inside of the virion correspond to Rev deleted size of genomic RNA. This suggests that retroviral RNA packaging is not a random process, and genomic RNA packaging occurs in the presence of Gag synthesis. The RNA bands on the northern blot are visualized by a radioactive probe that recognize the packaging sequence of HIV-1 (ψ).

### EXAMPLE 7

### M5 Gene Delivery Vector

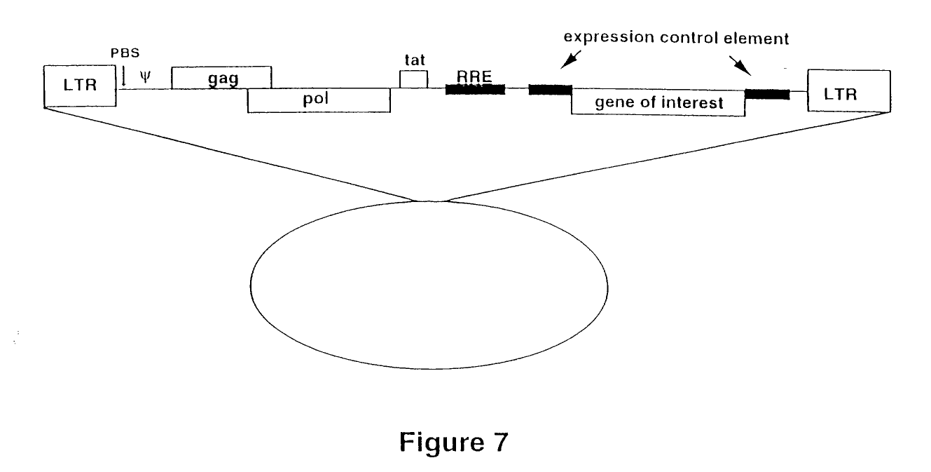

The genetic construct useful as a nucleic acid delivery vector comprising a retroviral packaging signal, a nucleotide sequence derived from viral RNA and which, in RNA form, is translatable to at least one protein and means to facilitate entry of a second nucleotide sequence and wherein said genetic construct, in an RNA form, is capable of being packaged in the presence of retroviral proteins provided in trans, is referred to as the “M5 gene delivery vector”. FIG. 7 is a diagrammatic representation of the M5 gene delivery vector.

The gene delivery retroviral particles produced by the expression of both the M5 gene delivery vector and the tropism determining vector (see Example 8) are more efficient in the packaging of genomic RNA and more efficient in gene delivery.

The M5 gene delivery vector in DNA form comprises all or a functional part of the following for efficient gene delivery:

(i) two retroviral long terminal repeat sequences (LTR) at the two ends of the retroviral DNA genome which are required for retroviral RNA expression. In an optional embodiment, a small deletion in the 3′LTR U3 region is also included to generate self inactive lentivirus, vector post gene delivery (FIGS. 9 and 28-32);

(ii) a primer binding site sequence (PBS) or equivalent required for the binding of retroviral primer tRNA for the synthesis of retroviral cDNA post viral entry into the target cells (FIG. 10);

(iii) a retroviral genomic RNA packaging sequence (q) or equivalent which is required for the packaging of genomic RNA during viral assembly (FIG. 11);

(iv) a nucleotide sequence derived from RNA and which, in an RNA form, is translatable to at least one protein (such as Gag, Gag-Pol and/or part of the retroviral Gag protein) [FIG. 12];

(v) optionally, a DNA sequence encoding a transcriptional enhancer element which is required to enhance the retroviral LTR driven transcription (FIG. 13);

(vi) a retroviral Rev responsive element (or equivalent) which is required for the nuclear retention of unspliced and singly spliced RNA in the absence of Rev protein (or equivalent) [FIG. 14];

(vii) a second nucleotide sequence (i.e. gene of interest) which consists of a genetic construct which is used for gene delivery into the target cells (FIG. 15); and/or

(viii) optionally, a gene of interest expression control element, such as a promoter sequence (which can be placed 5′ of the gene of interest) and/or a translational control sequence (which can be placed 3′ of the gene of interest) in order to enhance and/or for the conditional expression of gene of interest post gene delivery (FIG. 16).

### EXAMPLE 8

### Tropism Determining Vector

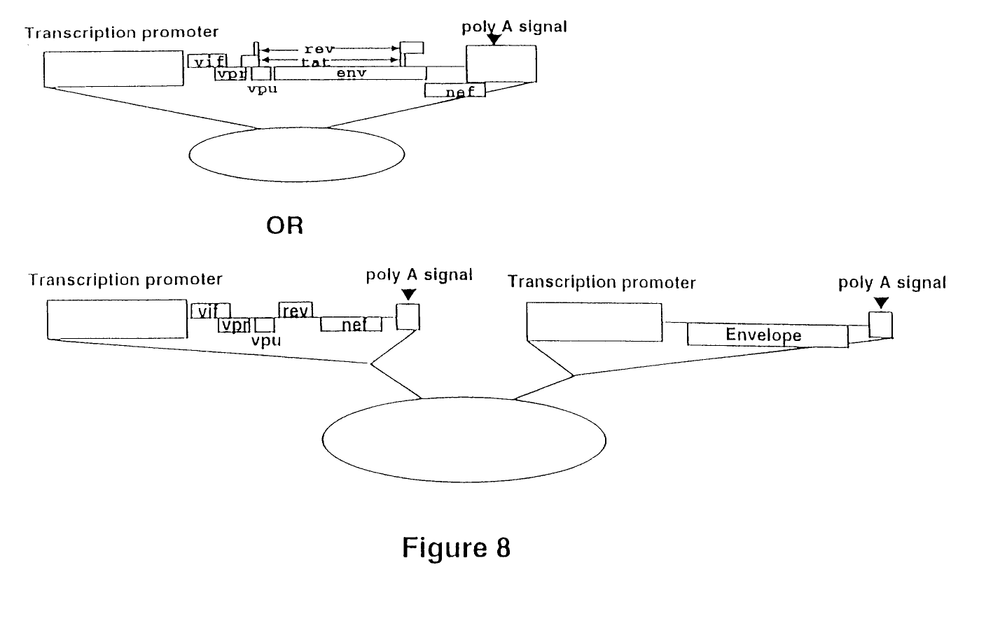

The vector which provides retroviral protein in trans for production of gene delivery retroviral particles via the M5 gene delivery vector (Example 7) is referred to as the “tropism determining vector”. FIG. 8 is a diagrammatic representation of the tropism determining vector. The tropism determining vector consists of all or a functional part of the following for the production of retroviral particles and efficient gene delivery:

(i) a nucleotide sequence, in an RNA form, translatable to a viral envelope protein as the envelope of M5 gene delivery vector (FIG. 17);

(ii) one or more nucleotide sequences, in RNA form, translatable to lentiviral auxiliary proteins Vif, Vpr and/or Nef and/or their equivalents or functional derivatives to enhance the gene delivery efficiency of the M5 gene delivery vector (FIG. 18), and/or

(iii) a nucleotide sequence, in an RNA form, translatable to lentiviral Rev protein (or equivalent) to facilitate the nucleus to cytoplasmic transport of the M5 gene delivery vector RNA for protein translation and genomic RNA packaging (FIG. 19).

### EXAMPLE 9

### Gene Delivery and Tropism Determining Vectors

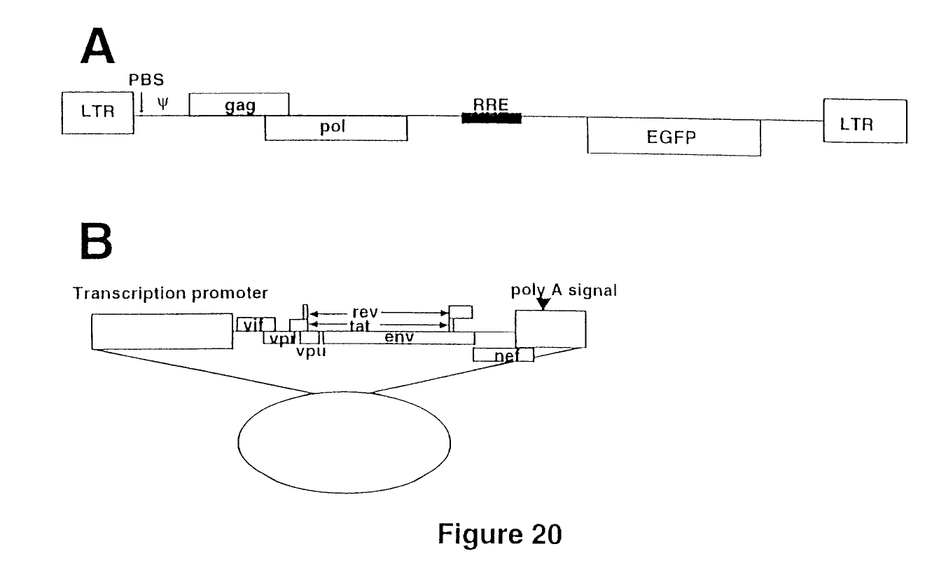

FIG. 20 is a diagrammatic representation of the prototypes of M5 gene delivery vector (A) and tropism determining vector (B) that were used in the present study. The prototype M5 gene delivery vector contains Gag. Gag-pol coding sequence, RRE coding sequence and green floresence protein (EGFP) as the gene of interest. The prototype tropism determining vector is essentially the second half of the HIV-1 genome that encodes for Env, Rev, Tat and HIV-1 auxiliary proteins (i.e. Vif, Vpr, Vpu and Nef).

### EXAMPLE 10

Conventional Retroviral Gene Delivery Vector with new Packaging System

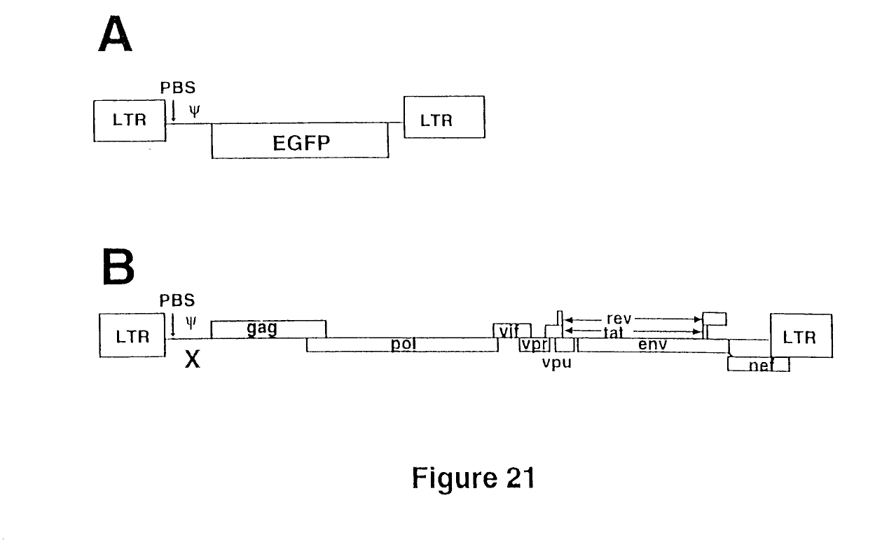

FIG. 21 is a diagrammatic representation of (A) a conventional retroviral gene delivery vector and (B) a plasmid DNA that provides viral proteins packaging cell line viral protein expression plasmid) for retroviral gene delivery vector. The conventional retroviral gene delivery vector does not contain any sequence to express functional viral proteins (such as Gag, Gag-Pol and Env), and the same green floresence protein (EGFP) as in the M5 gene delivery vector prototype is used as the gene of interest to compare for the efficiency of gene delivery between our design and the conventional vectors. The packaging cell line viral protein expression plasmid of the present invention contains a mutation at the packaging signal ψ which does not allow for the production of viral particles containing viral genomic RNA. However, upon transfection into a mammalian cell (such as 293T cells), the packaging cell line viral protein expression plasmid of the present invention produces all the necessary viral proteins for viral particles formation as a packaging cell line would.

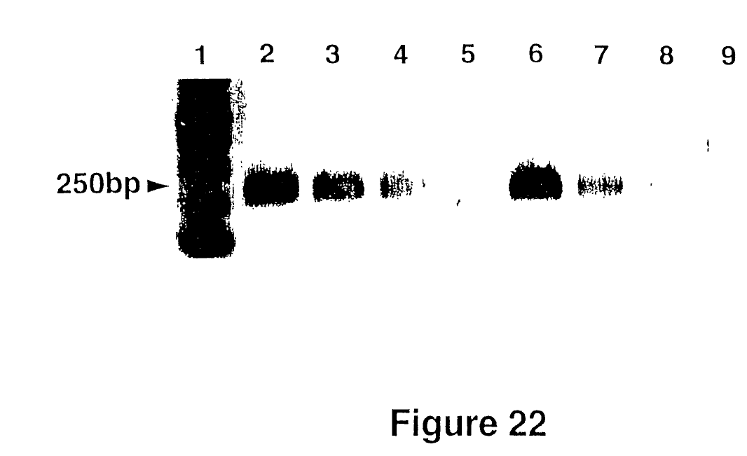

FIG. 22 is a photographic representation of the quantitative polymerase chain reaction (PCR) for cellular HLA-DQ a sequence—normalization of cellular DNA input for the comparison of gene delivery efficiency between conventional gene delivery vector and prototype of M5 gene delivery vector.

Conventional gene delivery retroviral particles were prepared by transfecting both plasmid DNA (conventional retroviral gene delivery vector plasmid (A) and packaging cell line viral protein expression plasmid (B)) shown in FIG. 21 into 293T cells via calcium phosphate transfection. Conventional gene delivery retroviral particles were isolated 36 hours post transfection. Prototype of MS gene delivery retroviral particles were prepared by transfecting both plasmid DNA (prototype of M5 gene delivery vector (A) and prototype of tropism determining vector (B)) shown in FIG. 20 into 293 T cell via calcium phosphate transfection. Prototype M5 gene delivery retroviral particles were isolated 36 hours post transfection. To ensure equivalent amount of conventional retroviral gene delivery particles and M5 gene delivery retroviral particles were used for our comparison study, quantitative p24 assay were used to measure the number of retroviral gene delivery particles (the PN as it is stated in page 10 under page 10 of detail description of the preferred embodiments) being produced in each transfection. Primary cells (PHA stimulated human peripheral blood mononuclear cells (PBMC)) were used as target cells for the comparison of these gene delivery vectors. Equivalent amount of conventional retroviral gene delivery particles and prototype M5 gene delivery retroviral particles were used to transduce (introduce gene of interest to the target cells) into same number of PBMCs. Cellular DNA were isolated at 24 and 48 hours post transduction Equivalent amount of cellular DNA were used to measure green floresence protein (EGFP) cDNA synthesis, and the quantity of EGFP cDNA were used as an indicator as the efficiency of transduction efficiency between these two types of gene delivery vector, i.e. since EGFP RNA (gene of interest) will be packaged into the gene delivery particles during viral assembly, but the EGFP cDNA will only be synthesized post virion entry into the target cells. Therefore, a stronger EGFP signal for the equivalent amount of HLA-DQa template will indicate a better gene delivery efficiency. PCR of EGFP DNA have also been done directly from the conventional retroviral gene delivery particles and the prototype M5 gene delivery retroviral particles to ensure the detected EGFP signal is not from contaminated DNA.

A 242 base pairs (bp) of HLA-DQ a gene was amplified in a PCR reaction using primers GH26 (5′gtgctgcaggtgtaaacttaccag3′ <400>1) and GH27 (5′cacggatccggtagcagcggtagagttg3′ <400>2) with 35 cycles of PCR reaction s. Serial dilution were used to ensure PCR amplification is within the linear range of the PCR amplification.

In FIG. 22, lane 1 is the 100 bp marker size DNA control. Lane 2, 3, 4 and 5 are dilution series of quantitative PCR reaction of HLA-DQ a of DNA extracted from PBMC donor C134 24 hours post transduction using the conventional retroviral gene delivery retroviral particle as described above under legend of FIG. 22. Lane 6, 7, 8 and 9 are dilution series of quantitative PCR reaction of HLA-DQ a of DNA extracted from PBMC donor C134 24 hours post transduction using the prototype M5 gene delivery retroviral particle as described above under legend of FIG. 22. The dilution series are—1:100 for lanes 2 & 6, 1:500 for lanes 3 & 7, 1:1000 for lanes 4 & 8, 1:5000 for lanes 5 & 9. Phosphorimaging and denstinometer were used to determine the intensity of the amplified PCR signal. Equivalent amount of cellular lysate were then used for the quantitative PCR for the EGFP signal.

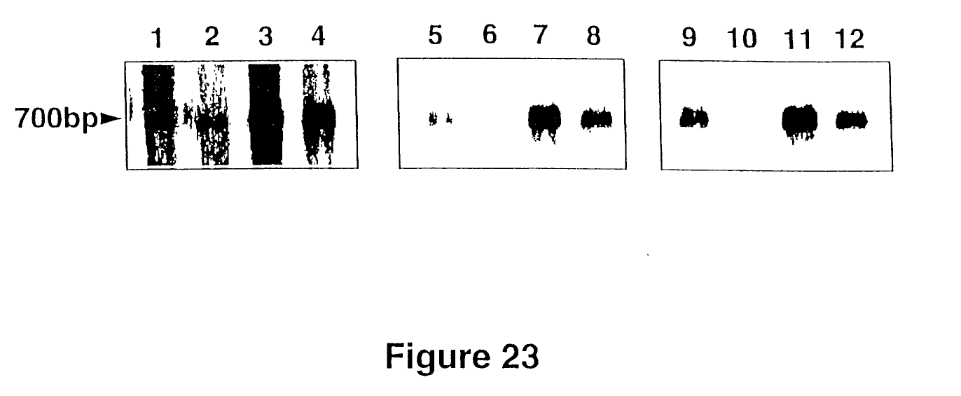

FIG. 23 is a photographic representation of the quantitative polymerase chain reaction (PCR) for EGFP cDNA synthesis using three different populations of donor PBMC as the target cells for gene delivery. A 700 bp long PCR fragment will be amplified in the presence of EGFP templates using primers EGFPs (5′acatgcatgctctagaatggtgagcaagggcgaggag3′ <400>3) and EGFPa (5′ccgctcgagttacttgtacagctcgtccatgcc3′ <400>4). Lanes 1 to 4, 5 to 8, 9 to 12 are from PBMC donors C134, E134 and K134, respectively. Lanes 1, 2, 5, 6, 9 and 10 are EGFP PCR amplification of PBMC lysates that were transduced with the conventional gene delivery retroviral particles. Lanes 3, 4, 7, 8, 11 and 12 are EGFP PCR amplification of PBMC lysates that were transduced with the prototype of M5 gene delivery retroviral particles. Lane 2 is a 1:2 template dilution of lane 1; lane 4 is a 1:2 template dilution of lane 3; lane 6 is a 1:2 template dilution of lane 5; lane 8 is a 1:2 template dilution of lane 7; lane 10 is a 1:2 template dilution of lane 9; lane 12 is a 1:2 template dilution of lane 11. Equivalent amount of cellular DNA (determined by quantitative HLA-DQα PCR) were used in each pair of quantitative EGFP PCR amplification respectively (lanes 1 & 3 pair, 2 & 4 pair, 5 & pair. 6 & 8 pair. 9 and 11 pair and 10 & 12 pair). The data clearly indicate that the M5 gene delivery retroviral particles are more efficient that the conventional gene delivery retroviral particles.

### EXAMPLE 11

### An HIV-1 Based M5 Gene Delivery Vector and Tropism Determining Vector Containing RRE Element and Transcription Enhancer Element

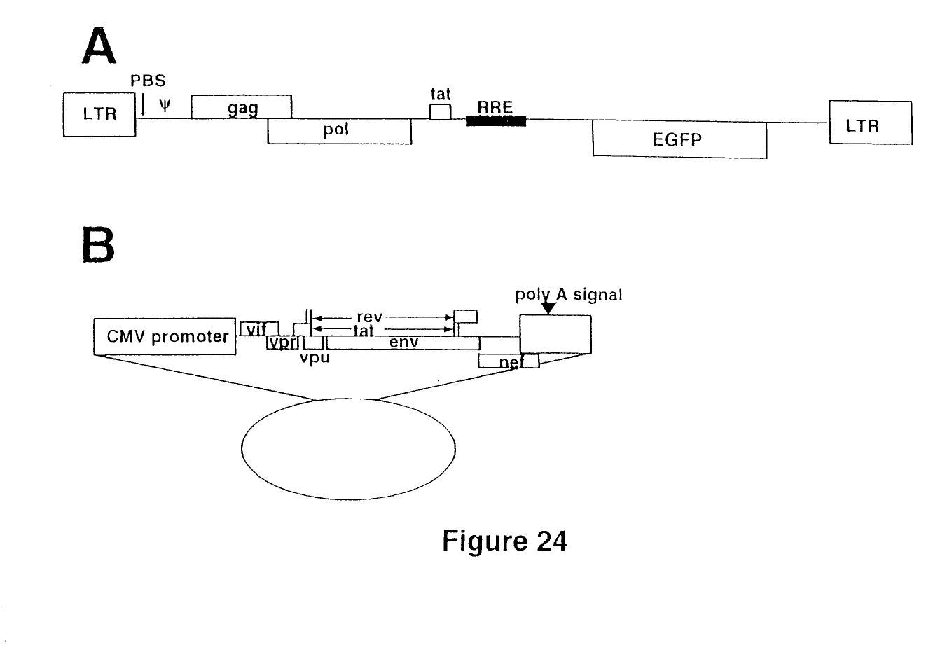

FIG. 24 is a diagrammatic representation of this prototype type. (A) is the HIV-1 based M5 gene delivery vector prototype. This M5 gene delivery vector consists of complete HIV-1 LTR sequences, HIV-1 primer binding site (PBS), HIV-1 packaging sequence (41), HIV-1 Gag and Gag-Pol coding sequence, HIV-1 transcription upregulation element Tat, HIV-1 mRNA nucleus retention element RRT and EGFP is an example for the gene of interest for this prototype. (B) is the HIV-1 based tropism determining vector. This tropism determining vector consists of HIV-1 envelope, coding sequence for HIV-1 auxiliary proteins Vif, Vpr, Vpu and Nef, and coding sequence for HIV-1 regulatory protein Tat and Rev. The expression of these viral protein is driven by a CMV immediately early promoter.

### EXAMPLE 12

### An HIV-2 Based M5 Gene Delivery Vector and HIV-1 Envelope Based Tropism Determining Vector Containing RRE Element and Transcription Enhancer Element

This second prototype is similar to the one described in Example 9 with the exception of using HIV-2 derived sequence for the M5 gene delivery vector. The different splicing control in HIV-2 is expected to further improve the packaging efficiency of gene of interest for gene delivery, HIV-1 tropism determining vector is used because of the higher spread of HIV-1 infection than HIV-2 infection would suggest HIV-1 envelope protein is more suitable for targeting the general population. A diagrammatic representation of this prototype type is also shown in FIG. 24. (A) is the HIV-2 based M5 gene delivery vector prototype. This M5 gene deliver, vector consists of complete HIV-2 LTR sequences. HIV-2 primer binding site (PBS). HIV-2 packaging sequence (ψ), HIV-2 Gag and Gag-Pol coding sequence. HIV-2 transcription upregulation element Tat. HIV-2 mRNA nucleus retention element RRE and EGFP is an example for the gene of interest for this prototype. (B) is the HIV-1 based tropism determining vector. This tropism determining vector consists of HIV-1 envelope, coding sequence for HIV-1 auxiliary proteins Vif, Vpr, Vpu and Nef, and coding sequence for HIV-1 regulatory protein Tat and Rev. The expression of these viral protein is driven by a CMV immediately early promoter.

### EXAMPLE 13

### A MoMuLV Based M5 Gene Delivery Vector and a VSV-G Envelope Based Tropism Determining Vector

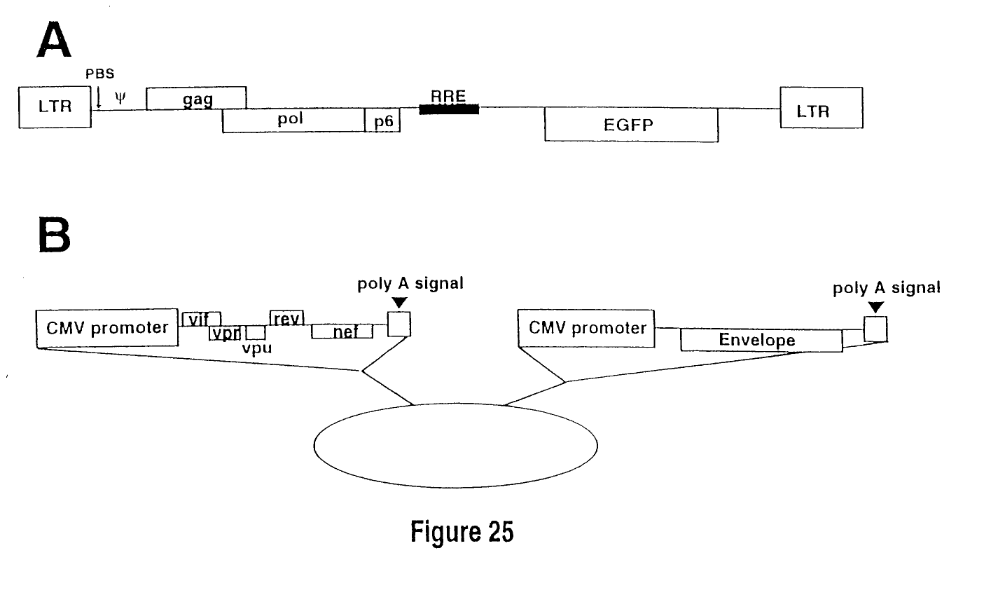

To avoid potential recombination events, a non-human pathogenic mouse retroviral gene delivery vector is constructed. FIG. 25 is a diagrammatic representation of this prototype type. (A) is a MoMuLV based M5 gene delivery vector that uses MoMuLV LTR, MoMuLV PBS, MoMuLV ψ sequence, MoMuLV Gag and Gag-Pol coding sequence, a HIV-1 p6 coding sequence is fused with the c terminus of the MoMuLV Pol coding sequence to facilitate the virion incorporation of HIV-1 Vpr, HIV-1 RRE sequence, and EGFP coding sequence as an example for the gene of interest. The incorporation of Vpr into MoMuLV based M5 gene delivery retroviral particle will allow genetic material to be delivered into non-dividing cells. The presence of HIV-1 RRE sequence will help to retain the unspliced mRNA into the nucleus to avoid unwanted immune response. (B) is the VSV-G envelope based tropism determining vector encodes for VSV-G protein as the source of viral envelope, that allows for wide range of gene delivery. The presence of HIV-1 viral auxiliary protein coding sequences (Vif, Vpr, Vpu and Nef) and coding sequence for regulatory protein Rev in the VSV-G envelope based tropism determining vector will enhance the transduction efficiency of these gene delivery retroviral particles. The expression of VSV-G envelope protein and HIV-1 auxiliary protein is under the control of CMV immediate early promoter.

### EXAMPLE 14

### A HIV-1 Based M5 Gene Delivery Vector with Internal Expression Control Element and Tropism Determining Vector Containing RRE Element and Transcription Enhancer Element

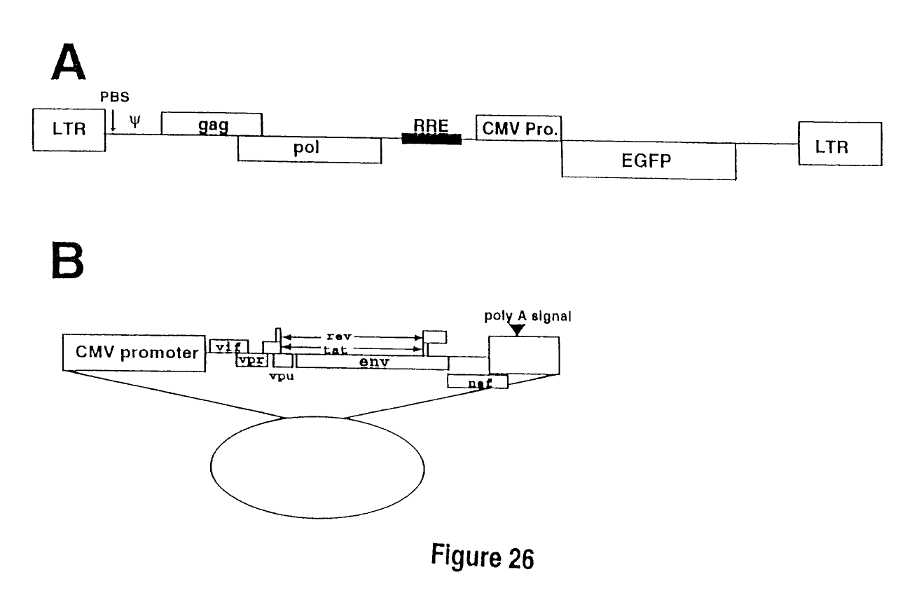

FIG. 26 is a diagrammatic representation of this prototype type. This design is similar to the prototype of Example 11 with the exception that the M5 gene delivery vector does not contain a Tat coding sequence and the expression of gene of interest post transduction is driven by an internal transcription promoter. (A) is the HIV-1 based M5 gene delivery vector prototype. This M5 gene delivery vector consists of complete HIV-1 LTR sequences, HIV-1 primer binding site (PBS), HIV-1 packaging sequence (ψ), HIV-1 Gag and Gag-Pol coding sequence, HIV-1 mRNA nucleus retention element RRE, a CMV immediate early promoter upstream of the gene of interest and EGFP is an example for the gene of interest for this prototype. (B) is the HIV-1 based tropism determining vector. This tropism determining vector consists of HIV-1 envelope, coding sequence for HIV-1 auxiliary proteins Vif, Vpr, Vpu and Nef, and coding sequence for HIV-1 regulatory protein Tat and Rev. The expression of these viral protein is driven by a CMV immediately early promoter.

### EXAMPLE 15

### An HIV-1 Based M5 Gene Delivery Vector with Internal Conditional Expression Control Element and Tropism Determining Vector Containing RRE Element and Transcription Enhancer Element

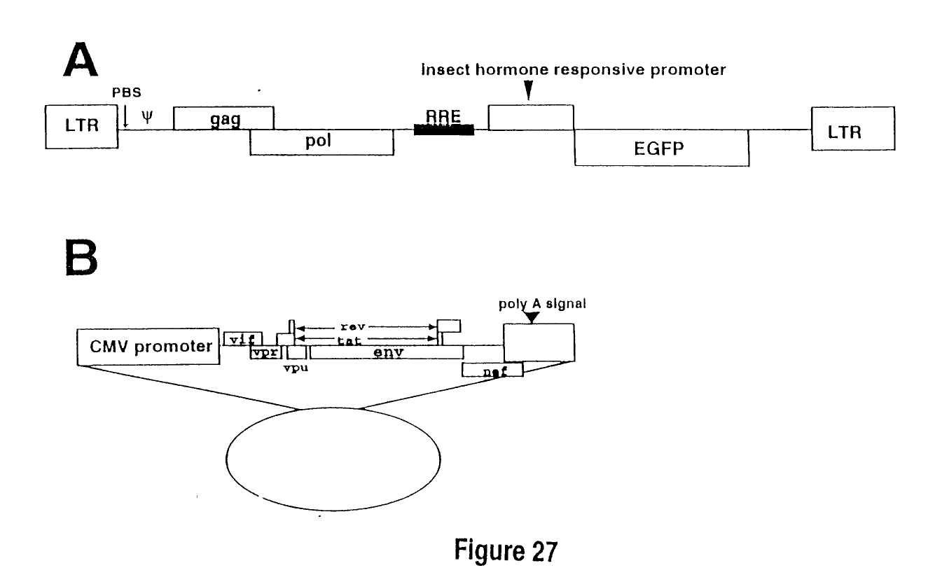

FIG. 27 is a diagrammatic representation of this prototype type. This design is similar to the prototype in Example 4 with the exception that the M5 gene delivery vector contains a conditional expression internal transcription promoter, such as ecdysone insect hormone responsive promoter instead of a CMV promoter. This allows for the gene of interest to be expressed upon stimulation post gene delivery. (A) is the HIV-1 based M5 gene delivery vector prototype. This M5 gene delivery vector consists of complete HIV-1 LTR sequences, HIV-1 primer binding site (PBS), HIV-1 packaging sequence (ψ), HIV-E Gag and Gag-Pol coding sequence. HIV-1 mRNA nucleus retention element RRE, a ecdysone insect hormone responsive promoter upstream of the gene of interest and EGFP is an example for the gene of interest for this prototype. (B) is the HIV-1 based tropism determining vector. This tropism determining vector consists of HIV-1 envelope, coding, sequence for HIV-1 auxiliary proteins Vif, Vpr, Vpu and Nef, and coding sequence for HIV-1 regulatory protein Tat and Rev. The expression of these viral protein is driven by a CMV immediately early promoter.

### EXAMPLE 16

### A MoMuLV Based M5 Gene Delivery Vector with Internal Expression Control Element and Self Inactivated LTR and a VSV-G Envelope Based Tropism Determining Vector

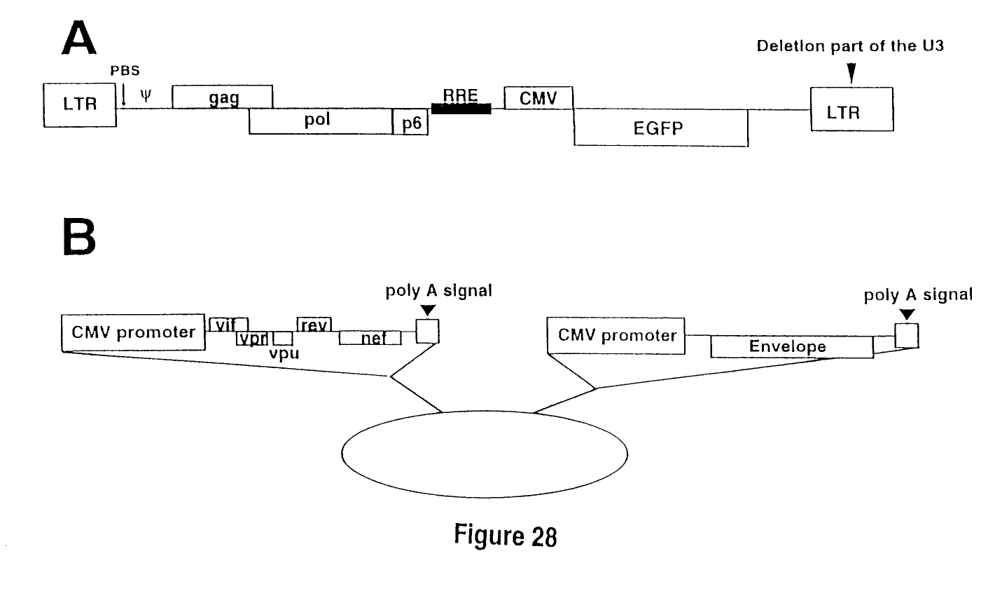

FIG. 28 is a diagrammatic representation of this prototype. This design is similar to the prototype of Example 13 with the exception that the M5 gene delivery vector contains a deletion of U3 sequence to generate a non-functional LTR post gene delivery and the M5 gene delivery vector has an internal expression control element. (A) is a MoMuLV based MS gene delivery vector that uses MoMuLV LTR, MoMuLV PBS, MoMuLV ψ sequence, MoMuLV Gag and Gag-Pol coding sequence, a HIV-1 p6 coding sequence is fused with the c terminus of the MoMuLV Pol coding sequence to facilitate the virion incorporation of HIV-1 Vpr, HIV-1 RRE sequence, a CMV immediate early promoter upstream of the gene of interest, EGFP coding sequence as an example for the gene of interest and a deletion of the U3 sequence to inactivate the LTR post gene delivery. The incorporation of Vpr into MoMuLV based M5 gene delivery retroviral particle will allow genetic material to be delivered into non-dividing cells. The presence of HIV-1 RRE sequence AND self inactivate LTR will help to retain the unspliced mRNA into the nucleus to avoid unwanted immune response. (B) is the VSV-G envelope based tropism determining vector encodes for VSV-G protein as the source of viral envelope, that allows for wide range of gene delivery. The presence of HIV-1 viral auxiliary protein coding sequences (Vif, Vpr, Vpu and Nef) and coding sequence for regulatory protein Rev in the VSV-G envelope based tropism determining vector will enhance the transduction efficiency of these gene delivery retroviral particles. The expression of VSV-G envelope protein and HIV-1 auxiliary protein is under the control of CMV immediate early promoter.

### EXAMPLE 17

### A MoMuLV Based M5 Gene Delivery Vector with Internal Conditional Expression Control Element and Self Inactivated LTR and a VSV-G Envelope Based Tropism Determining Vector

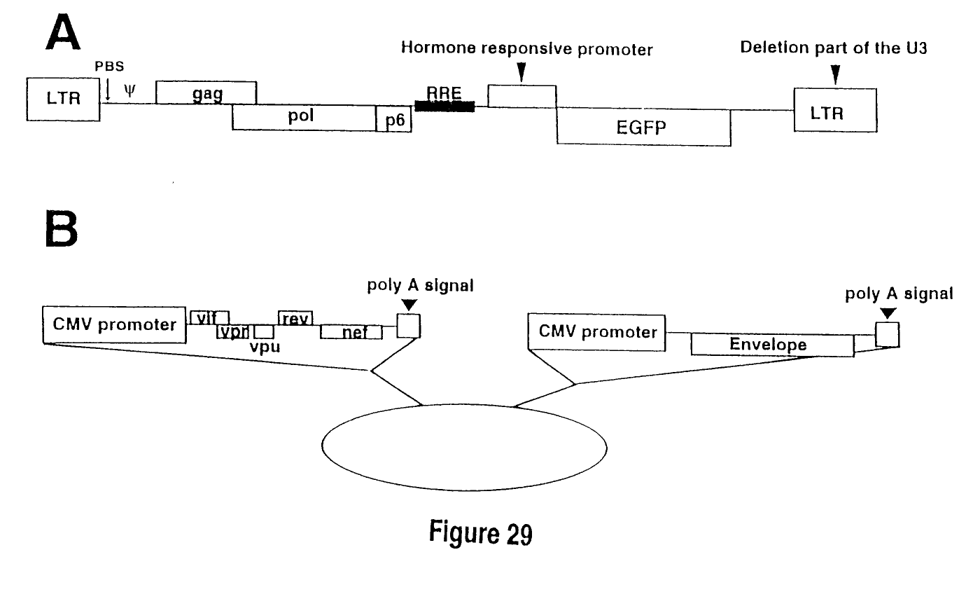

FIG. 29 is a diagrammatic representation of this prototype. This design is similar to the prototype in Example 16 with the exception that the M5 gene delivery vector contains an internal conditional expression control element. (A) is a MoMuLV based M5 gene delivery vector that uses MoMuLV LTR, MoMuLV PBS, MoMuLV ψ sequence, MoMuLV Gag and Gag-Pol coding sequence, a HIV-1 p6 coding sequence is fused with the c terminus of the MoMuLV pol coding sequence to facilitate the virion incorporation of HIV-1 Vpr, HIV-1 RRE sequence, an ecdysone insect hormone responsive promoter upstream of the gene of interest, EGFP coding sequence as an example for the gene of interest and a deletion of the U3 sequence to inactivate the LTR post gene delivery. (B) is the VSV-G envelope based tropism determining vector encodes for VSV-G protein as the source of viral envelope, that allows for wide range of gene delivery. The presence of HIV-1 viral auxiliary protein coding sequences (Vif, Vpr, Vpu and Nef) and coding sequence for regulatory protein Rev in the VSV-G envelope based tropism determining vector enhances the transduction efficiency of these gene delivery retroviral particles. The expression of VSV-G envelope protein and HIV-1 auxiliary protein is under the control of CMV immediate early promoter.

### EXAMPLE 18

### A MoMuLV Based M5 Gene Delivery Vector with Internal Conditional Expression Control Element and Self Inactivated LTR and a Hepatitis Virus Envelope Based Tropism Determining Vector

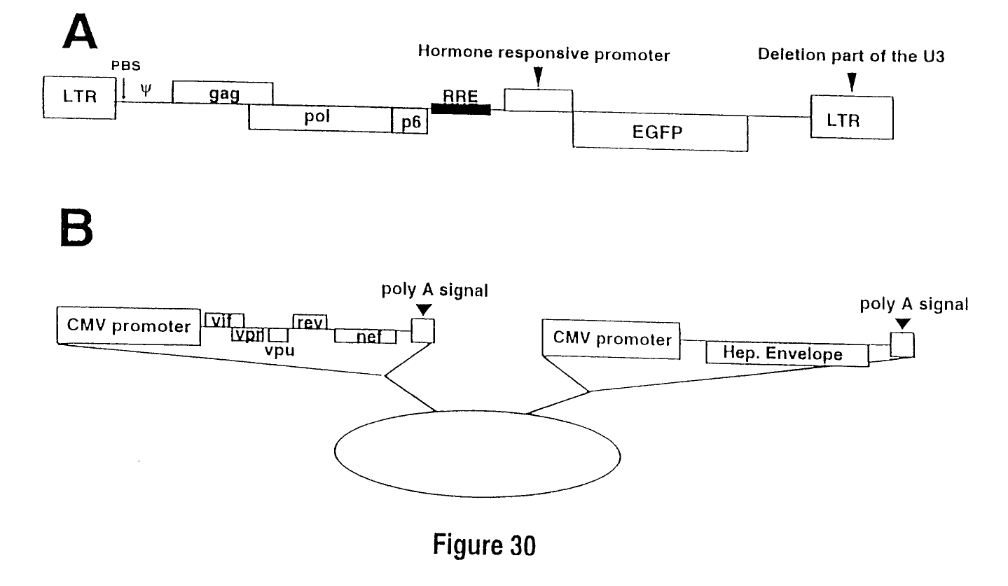

FIG. 30 is a diagrammatic representation of this prototype. This design is similar to the prototype of Example 17 with the exception that the tropism determining vector encodes hepatitis viral envelope instead of VSV-G envelope. (A) is a MoMuLV based M5 gene delivery vector that uses MoMuLV LTR. MoMuLV PBS. MoMuLV ψ sequence. MoMuLV Gag and Gag-Pol coding sequence, a HIV-1 p6 coding sequence is fused with the c terminus of the MoMuLV pol coding sequence to facilitate the virion incorporation of HIV-1 Vpr, HIV-1 RRE sequence, an ecdysone insect hormone responsive promoter upstream of the gene of interest, EGFP coding sequence as an example for the gene of interest and a deletion of the U3 sequence to inactivate the LTR post gene delivery. (B) is the hepatitis virus envelope based tropism determining vector encodes for hepatitis virus envelope protein as the source of viral envelope, which allows for specific targeting of MS gene delivery retroviral gene delivery vector to liver cell type for gene delivery. Similar approach can be used to target M5 gene delivery vector to various cell types by altering the envelope coding sequence in the tropism determining vector. The presence of HIV-1 viral auxiliary protein coding sequences (Vif, Vpr, Vpu and Nef) and coding sequence for regulatory protein Rev in the hepatitis virus envelope based tropism determining vector will enhance the transduction efficiency of these gene delivery retroviral particles. The expression of hepatitis virus envelope protein and HIV-1 auxiliary protein is under the control of CMV immediate early promoter.

### EXAMPLE 19

### A MoMuLV Based M5 Gene Delivery Vector with Internal Conditional Expression Control Element and Self Inactivated LTR and a Hepatitis Virus Envelope Based Tropism Determining Vector

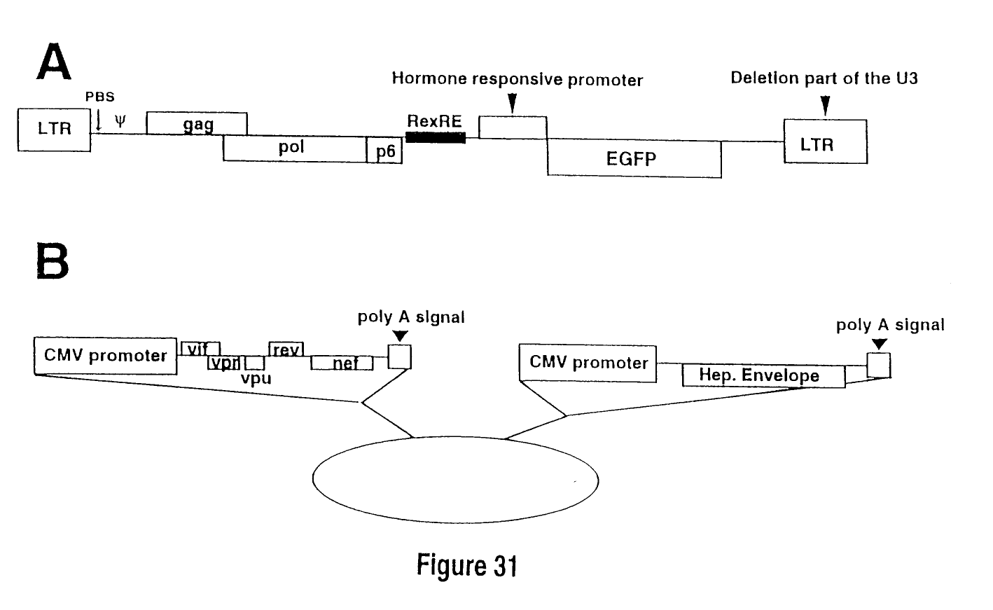

FIG. 31 is a diagrammatic representation of this prototype. This design is similar to the prototype of Example 18 with the exception chat the M5 gene delivery vector codes for HTLV-1 Rex responsive element instead of HIV-1 RRE. (A) is a MoMuLV based M5 gene delivery vector that uses MoMuLV LTR, MoMuLV PBS. MoMuLV ψ sequence, MoMuLV Gag and Gag-Pol coding sequence, a HIV-1 p6 coding sequence is fused with the c terminus of the MoMuLV pol coding sequence to facilitate the virion incorporation of HIV-1 Vpr, HTLV-1 RexRE sequence, an ecdysone insect hormone responsive promoter upstream of the gene of interest, EGFP coding sequence as an example for the gene of interest and a deletion of the U3 sequence to inactivate the LTR post gene delivery (B) is the hepatitis virus envelope based tropism determining vector encodes for hepatitis virus envelope protein as the source of viral envelope, which allows for specific targeting of M5 gene delivery retroviral gene delivery vector to liver cell type for gene delivery. Similar approach can be used to target M5 gene delivery vector to various cell types by altering the envelope coding sequence in the tropism determining vector. The presence of HTV-1 viral auxiliary protein coding sequences (Vif, Vpr, Vpu and Nef) and coding sequence for regulatory protein Rev in the hepatitis virus envelope based tropism determining vector will enhance the transduction efficiency of these gene delivery retroviral particles. The expression of hepatitis virus envelope protein and HIV-1 auxiliary protein is under the control of CMV immediate early promoter.

### EXAMPLE 20

### A MoMuLV Based M5 Gene Delivery Vector with Internal Conditional Expression Control Element and Self Inactivated LTR and a Hepatitis Virus Envelope Based Tropism Determining Vector

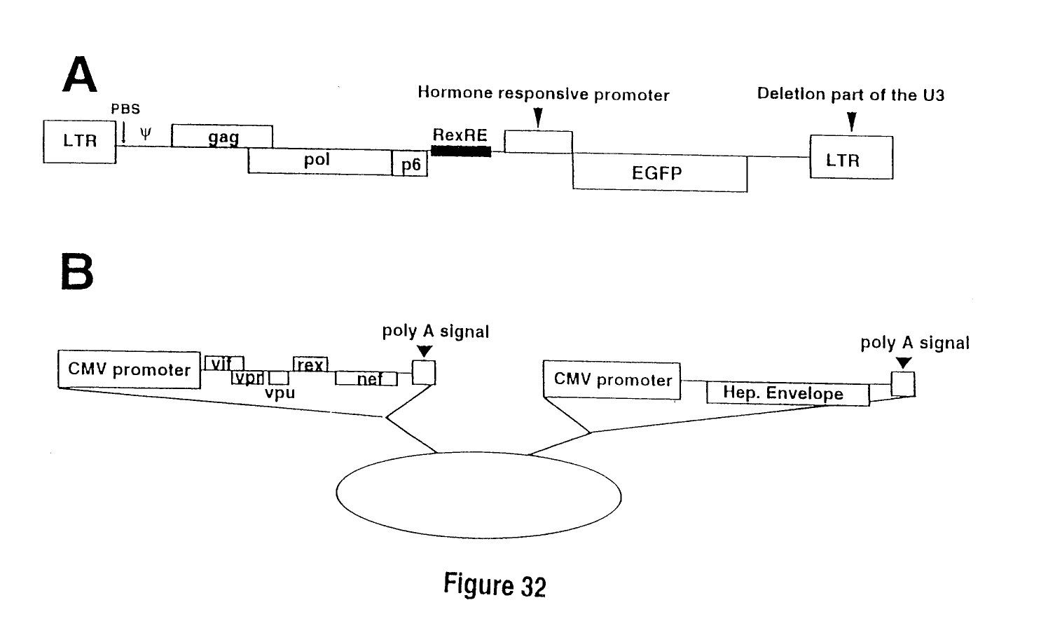

FIG. 32 is a diagrammatic representation of this prototype. This design is similar to the prototype of Example 19 with the exception that the envelope tropism determining vector codes for HTLV-1 Rex instead of HIV-1 Rev. (A) is a MoMuLV based M5 gene delivery vector that uses MoMuLV LTR. MoMuLV PBS, MoMuLV ψ sequence, MoMuLV Gag and Gag-Pol coding sequence, a HIV-1 p6 coding sequence is fused with the c terminus of the MoMuLV pol coding sequence to facilitate the virion incorporation of HIV-1 Vpr, HTLV-1 RexRE sequence, an ecdysone insect hormone responsive promoter upstream of the gene of interest, EGFP coding sequence as an example for the gene of interest and a deletion of the U3 sequence to inactivate the LTR post gene delivery. (B) is the hepatitis virus envelope based tropism determining vector encodes for hepatitis virus envelope protein as the source of viral envelope, which allows for specific targeting of M5 gene delivery retroviral gene delivery vector to liver cell type for gene delivery. Similar approach can be used to target M5 gene delivery vector to various cell types by altering the envelope coding sequence in the tropism determining vector. The presence of HIV-1 viral auxiliary protein coding sequences (Vif, Vpr, Vpu and Nef) and coding sequence for regulatory protein HTLV-1 Rex in the hepatitis virus envelope based tropism determining vector will enhance the transduction efficiency of these gene delivery retroviral particles. The expression of hepatitis virus envelope protein and HIV-1 auxiliary protein is under the control of CMV immediate early promoter.

Those skilled in the art will appreciate that the invention described herein is susceptible to variations and modifications other than chose specifically described. It is to be understood that the invention includes all such variations and modifications. The invention also includes all of the steps, features, compositions and compounds referred to or indicated in this specification, individually or collectively, and any and all combinations of any two or more of said steps or features.

TABLE 1
RT ACTIVITY OF THE CO-TPANSFECTION SYSTEM

| | Plasmid DNA | RT ACTIVITY IN TRIPLICATE | | |
|---|---|---|---|---|
| Cell Types Used | Used | (cpm/10 μl) | | |
| 293T | Rev- | 361 | 319 | 291 |
| | FS/P[-] | 425 | 368 | 481 |
| | Rev-& FS/P[-] | 7297 | 6832 | 8379 |
| COS | Rev- | 25 | 23 | 24 |
| | FS/P[-] | 32 | 26 | 54 |
| | Rev-& FS/P[-] | 1913 | 1628 | 1939 |
| Media/Background | | 34 | 25 | 22 |

### BIBLIOGRAPHY

1. Coffin 1992a In The retroviridae (ed. J. A. Levy) p.p. 19-49. Plenum Press, New York.

2. Coffin 1992b Current Topics in Microbiology and Immunology 176.143-164.

3. Coffin 1996 In Virology (ed. B. N. Fields et al) pp 1767-1848 Raven Press. New York.

4. Mann et al, 1983 Cell 32: 871-879

5. Watanabe & Temin 1983 Mol. Cell. Biol. 3: 2241-2249

6. Miller & Rosman, 1989 BioTechniques 7: 980-990.

1. A genetic construct useful as a nucleic acid delivery vector comprising a retroviral packaging signal, a nucleotide sequence derived from viral RNA and which, in RNA form, is translatable to at least one protein and means to facilitate entry of a second nucleotide sequence and wherein said genetic construct, in an RNA form, is capable of being packaged in the presence of retroviral proteins provided in trans with greater efficiency than a genetic construct comprising a packaging signal and said second nucleotide sequence without a nucleotide sequence derived from a retrovirus and which, in RENA form, is capable of translation into a viral protein.

2. A genetic construct according to claim 1 wherein the nucleotide sequence translatable to at least one protein encompasses the gag coding region of a retrovirus.

3. A genetic construct according to claim 1 wherein the nucleotide sequence translatable to at least one protein encompasses the gag and pol coding region of a retrovirus.

4. A genetic construct according to claim 1 wherein means to facilitate entry of the second nucleotide sequence is restriction endonuclease digestion followed by ligation.

5. A genetic construct according to claim 4 wherein means to facilitate entry of a second nucleotide sequence is cleavage of a restriction endonuclease site followed by ligation of the second nucleotide sequence into the site of cleavage.

6. A genetic construct according to claim 1 wherein the nucleic acid delivery vector lacks a functional env coding sequence.

7. A genetic construct according to claim 1 or 6 wherein the nucleic acid delivery vector lacks a functional rev coding sequence.

8. A genetic construct according to claim 1 wherein the second nucleotide sequence corresponds to a gene, anti-sense molecule, co-suppression molecule or ribozyme.

9. A genetic construct useful as a nucleic acid delivery vector comprising a nucleotide sequence derived from viral RNA and which, in RNA form, is translatable to at least one protein or derivative thereof encoded by all or part of the gag region of a retrovirus and means to facilitate entry of a second nucleotide sequence.

10. A genetic construct useful as a nucleic acid delivery vector comprising a nucleotide sequence derived from viral RNA and which, in RNA form, is translatable to at least one protein or derivative thereof encoded by all or part of the gag region of a retrovirus and means to facilitate entry of a second nucleotide sequence and wherein said genetic construct, in an RNA form, is capable of being packaged in the presence of retroviral proteins provided in trans with greater efficiency than a genetic construct comprising a packaging signal and said second nucleotide sequence without a nucleotide sequence derived from a retrovirus and which, in RNA form, is capable of translation into at least one protein or derivative thereof encoded by the gag region.

11. A nucleic acid packaging system comprising at least one vector wherein a first vector comprises a retroviral packaging signal, a nucleotide sequence derived from viral RNA and which, in RNA form, is translatable to at least one protein and means to facilitate entry of a second nucleotide sequence and wherein said genetic construct, in an RNA form, is capable of being packaged in the presence of retroviral proteins provided in trans with greater efficiency than a genetic construct comprising a packaging signal and said second nucleotide sequence without a nucleotide sequence derived from a retrovirus and which, in RNA form, is capable of translation into a viral protein, said retroviral packaging system further comprising at least one other vector or packaging cell wherein said vector or cell's genome comprises at least one other retroviral coding region or a functional derivative thereof.

12. A nucleic acid packaging system comprising at least one vector wherein a first vector comprises a retroviral packaging signal, a nucleotide sequence derived from viral RNA and which, in RNA form is translatable to at least one protein and means to facilitate entry of a second nucleotide sequence and wherein said genetic construct, in an RNA form, is capable of being packaged in the presence of retroviral proteins provided in trans with greater efficiency than a genetic construct comprising a packaging signal and said second nucleotide sequence without a nucleotide sequence derived from a retrovirus and which, in RNA form, is capable of translation into a viral protein; said retroviral packaging system further comprising at least one other vector or packaging cell wherein said vector of cell's genome comprises the env region of a virus or a functional equivalent or derivative thereof.

13. A nucleic acid packaging system comprising a First and second vector wherein the first vector is a nucleic acid delivery vector and comprises a retroviral packaging signal, a nucleotide sequence derived from viral RNA and which, in RNA form, is translatable to at least one protein encoded by the gag region of a retrovirus and means to facilitate entry of a second nucleotide sequence; wherein said second vector comprises the env region of a virus or a functional equivalent or derivative thereof.

14. A nucleic acid packaging system comprising a nucleic acid delivery vector having a retroviral packaging signal, a nucleotide sequence derived from viral RNA and which, in RNA form, is translatable to at least one protein encoded by the gag region of a retrovirus and means to facilitate entry of a second nucleotide sequence and wherein said genetic construct, in an RNA form, is capable of being packaged in the presence of retroviral proteins provided in trans with greater efficiency than a genetic construct comprising a packaging signal and said second nucleotide sequence without a nucleotide sequence derived from a retrovirus and which, in RNA form, is capable of translation into a protein encoded by the gag region of a retrovirus gag protein, and at least one other vector comprising the env coding region of a virus or a functional equivalent or derivative thereof.

15. A retroviral packaging system according to claim 14 wherein said nucleic acid delivery vector encodes at least one Gag protein and at least one Pol protein or functional derivatives thereof.

16. A retroviral packaging system according to claim 14 or 15 wherein the nucleic acid delivery vector comprises the wild-type gag and pol regions of one or more retroviruses.

17. A retroviral packaging system according to claim 14 or 15 or 16 wherein said nucleic acid delivery vector encodes a Rev responsive element (RRE) but does not contain a functional rev.

18. A method for packaging a nucleic acid, said method comprising introducing said nucleic acid into a nucleic acid delivery vector comprising a retroviral packaging signal and a nucleotide sequence derived from viral RNA and which, in RNA form, is translatable to at least one protein and wherein said genetic construct, in an RNA form, is capable of being packaged in the presence of retroviral proteins provided in trans with greater efficiency than a genetic construct comprising a packaging signal and said gene without a nucleotide sequence derived from a retrovirus and which, in RNA form, is capable of translation into a viral protein; introducing said nucleic acid delivery vector into a cell wherein said cell is capable of providing the genetic material or a protein encoded thereby in trans to permit packaging of said gene into a virion.

19. A method according to claim 18 wherein at least two vectors are used wherein one vector is a nucleic acid delivery vector and at least one other vector carries genetic material required for packaging.

20. A method according to claim 19 wherein the second vector carries all or a functional part of env.

21. A method according to claim 19 or 20 wherein the first vector comprising a nucleic acid delivery vector further comprises (i) gag or a region substantially corresponding to gag, (ii) optionally pol or a region substantially corresponding to pol; and (iii) a gene of interest.

22. A method for packaging a nucleic acid of interest, said method comprising introducing said nucleic acid into a nucleic acid delivery vector comprising a retroviral packaging signal, gag or a functionally equivalent region thereof and optionally pol or a functionally equivalent region thereof corresponding to pol and introducing said nucleic acid delivery vector into a cell which contains or is capable of containing genetic material or a protein encoded thereby to permit packaging of said gene of interest into a virion.

23. A method according to claim 22 wherein the genetic information required for packaging is provided on at least one plasmid wherein at least one plasmid carries an env region from a retrovirus or a functional equivalent or derivative thereof.

24. Use of the gag region of a retrovirus or a functionally equivalent region thereof in the manufacture of a nucleic acid delivery vector capable of carrying a nucleic acid of interest to be packaged.

25. A gene delivery vector in DNA form comprising all or a functional part of the following:
(i) two retroviral long terminal repeat sequences (LTR) at the two ends of the retroviral DNA genome;
(ii) a primer binding site sequence (PBS);
(iii) a retroviral genomic RNA packaging sequence (ψ) or equivalent;
(iv) a nucleotide sequence derived from RNA and which, in an RNA form, is translatable to at least one protein;
(v) optionally, a DNA sequence encoding a transcriptional enhancer element;
(vi) a retroviral Rev responsive element or its equivalent;
(vii) a second nucleotide sequence which consists of a genetic construct that is used for gene delivery into the target cells (e.g. see FIG. 15); and/or


SEQUENCE LISTING (4 sequences)

SEQ ID NO: 1 - DNA, 33 bp, Artificial Sequence
  synthetic construct
ccgctcgagttacttgtacagctcgtccatgcc

SEQ ID NO: 2 - DNA, 28 bp, Artificial Sequence
  synthetic construct
cacggatccggtagcagcggtagagttg

SEQ ID NO: 3 - DNA, 37 bp, Artificial Sequence
  synthetic construct
acatgcatgctctagaatggtgagcaagggcgaggag

SEQ ID NO: 4 - DNA, 33 bp, Artificial Sequence; identical to SEQ ID NO: 1
  synthetic construct